(19) United States
(12) Patent Application Publication
(10) Pub. No.: US 2022/0341861 A1
(43) Pub. Date: Oct. 27, 2022

(54) Thermodynamic Formulation for Langmuir Adsorption Isotherms

(71) Applicant: Texas Tech University System, Lubbock, TX (US)

(72) Inventor: Chau-Chyun Chen, Lubbock, TX (US)

(21) Appl. No.: 17/616,287

(22) PCT Filed: Aug. 10, 2020
(86) PCT No.: PCT/US2020/045586

§ 371 Date: Dec. 3, 2021

Related U.S. Application Data
(60) Provisional application No. 62/860,319, filed on Jun. 12, 2019.

(51) Int. Cl. G01N 25/48

(52) U.S. Cl. CPC G01N 25/4806

(57) ABSTRACT

The present invention includes a method for thermodynamic formulation of a Langmuir isotherm comprising: (1), (1′) (1), (1′) where nᵢ is the adsorption amount of gas component i; (1′) is the adsorption maximum amount; P is the gas vapor pressure, and K is the apparent adsorption equilibrium constant in which adsorption and desorption rates are proportional to a concentrations of vacant sites and occupied sites; and substituting the concentration of both a vacant site and an occupied site with site activities, wherein a reference state for the vacant sites is at zero surface coverage while the reference state for the occupied sites is at full surface coverage.

$$\begin{matrix}( {1}^{′} ) & \\ {n}_{i} = {n}_{i}^{0} \frac{KP}{1 + KP} & ( 1 )\end{matrix}$$

### CROSS-REFERENCE TO RELATED APPLICATIONS

This application claims priority to U.S. Provisional Application Serial No. 62/860,319, filed Jun. 12, 2019, the entire contents of which are incorporated herein by reference.

### STATEMENT OF FEDERALLY FUNDED RESEARCH

This invention was made with government support under DE-EE0007888 awarded by the U.S. Department of Energy. The government has certain rights in the invention.

### TECHNICAL FIELD OF THE INVENTION

The present invention relates in general to the field of thermodynamic modeling, and more particularly, to a thermodynamic formulation for Langmuir adsorption isotherms that improves on the currently available calculations.

### BACKGROUND OF THE INVENTION

Without limiting the scope of the invention, its background is described in connection with classical Langmuir isotherm modeling.

The classical Langmuir isotherm model [8] is considered the first scientifically sound expression for pure component adsorption isotherms:

$${n}_{i} = {n}_{i}^{0} \frac{KP}{1 + KP} \qquad ( 1 )$$

where nᵢ is the adsorption amount of gas component i; nᵢ⁰ is the adsorption maximum amount; and P is the gas vapor pressure. Indicative of the affinity between adsorbate and adsorbent, K is the apparent adsorption equilibrium constant. The Langmuir isotherm has been extensively used to describe adsorption behavior of many systems including adsorption of non-polar 1gases on activated carbons and zeolites. Ignoring the surface heterogeneity and the van der Waals interactions between adsorbates and adsorbents [9, 10], the Langmuir isotherm is inadequate in describing pure component adsorption isotherms especially at low temperature and high pressure regions [11].

Among the many efforts [12-14] to improve upon the classical Langmuir isotherm model, the empirical Sips isotherm model [12, 13] is probably the most successful. Following Freundlich isotherm [15, 16], Sips introduced an empirical “heterogeneity” parameter m, which is usually less than unity [17], to the Langmuir isotherm. Shown in Eq. 2, the resulting Sips isotherm expression is much more flexible in representing adsorption isotherm data.

$${n}_{i} = {n}_{i}^{0} \frac{{( KP )}^{m}}{1 + {( KP )}^{m}} \qquad ( 2 )$$

With three adjustable parameters (nᵢ⁰, K and m), the Sips isotherm expression and other similar empirical expressions are capable of correlating pure component adsorption isotherm data much better than the Langmuir isotherm could achieve with two adjustable parameters (nᵢ⁰ and K). However, the introduction of empirical heterogeneity parameter m distorts the theoretical basis of the classical Langmuir isotherm and the physical significance of the Langmuir isotherm parameters (nᵢ⁰ and K) is lost.

What is needed are novel methods for calculating Langmuir isotherms that have a higher correlation with empirically measured isotherms.

### SUMMARY OF THE INVENTION

In one embodiment, the present invention includes a method for thermodynamic formulation of a Langmuir isotherm comprising:

$${n}_{i} = {n}_{i}^{0} \frac{KP}{1 + KP} \qquad ( 1 )$$

where nᵢ is the adsorption amount of gas component i; nᵢ⁰ is the adsorption maximum amount; P is the gas vapor pressure, and K is the apparent adsorption equilibrium constant in which adsorption and desorption rates are proportional to a concentrations of vacant sites and occupied sites; and substituting the concentration of both a vacant site and an occupied site with site activities, wherein a reference state for the vacant sites is at zero surface coverage while the reference state for the occupied sites is at full surface coverage. In one aspect, the method further comprises substituting the constant K with a thermodynamic adsorption equilibrium constant K° calculated:

$$K° = \frac{{k}_{a}}{{k}_{d}} = \frac{{a}_{AS}}{P {a}_{S}} = \frac{{γ}_{1} {x}_{1}}{{γ}_{ϕ} ( 1 - {x}_{1} ) P} \qquad ( 6 )$$

wherein α_{AS} is the activity of a site occupied with an adsorbed gas A, α_{S} is an activity of the vacant site, γ₁ and γ_{ϕ} are an activity coefficient of the occupied site with adsorbed gas component 1 and an activity coefficient of the vacant site, respectively. In another, aspect the reference state for a vacant site is chosen to be at zero surface coverage, wherein, γ₁=1 at x₁=1, and γ_{ϕ}=1 at x₁=0. In another aspect, the method further comprises reformulating Eq. 6, one obtains the following implicit adsorption isotherm expression:

$${n}_{1} = {n}_{1}^{0} \frac{K° {γ}_{ϕ} P}{{γ}_{1} + K° {γ}_{ϕ} P} \qquad ( 7 )$$

wherein γ₁ and γ_{ϕ} are functions of x₁ and a relationship between the thermodynamic adsorption equilibrium constant K° and the apparent adsorption equilibrium constant K is shown in Eq. 8.

$$K ( {x}_{1} ) = K° \frac{{γ}_{ϕ} ( {x}_{1} )}{{γ}_{1} ( {x}_{1} )} . \qquad ( 8 )$$

In another aspect, the method further comprises calculating one or more pure component isotherms for gases with adsorbents including silica gels, activated carbons, zeolites and metal organic frameworks. In another aspect, the method further comprises calculating one or more pure component isotherms for gases with adsorbents including silica gels, activated carbons, zeolites and metal organic frameworks at one or more temperatures. In another aspect, the site activities are further calculated with an adsorption Non-Random Two-Liquid (aNRTL) activity coefficient. In another aspect, a reference state for an occupied site with adsorbed gas component 1 is at full surface coverage and a saturated adsorption state is x₁=1. In another aspect, the method further comprises substituting the species concentrations with the species activities and calculates the species activity coefficients with the adsorption Non-Random Two-Liquid activity coefficient. In another aspect, an adsorption equilibria calculated is at least one of: thermodynamically consistent; requires few adjustable model parameters; is applicable to both pure component adsorption isotherms and multicomponent adsorption isotherms; or calculates multicomponent adsorption isotherms from pure component adsorption isotherms.

In another embodiment, the present invention includes a method of determining adsorption isotherms for at least one of: a first temperature, a first pressure, a low temperature, or a high pressure region, or both comprising:

$${n}_{1} = {n}_{1}^{0} \frac{K° {γ}_{ϕ} P}{{γ}_{1} + {K° γ}_{ϕ} P} ; and$$

$$K° = \frac{{k}_{a}}{{k}_{d}} = \frac{{a}_{A S}}{P {a}_{S}} = \frac{{γ}_{1} {x}_{1}}{{γ}_{ϕ} ( 1 - {x}_{1} ) P}$$

where nᵢ is the adsorption amount of gas component i; nᵢ⁰ is the adsorption maximum amount; P is the gas vapor pressure, α_{AS} is the activity of a site occupied with an adsorbed gas A, α_{S} is an activity of the vacant site, γ₁ and γ_{ϕ} are an activity coefficient of the occupied site with adsorbed gas component 1 and an activity coefficient of the vacant site, respectively. In one aspect, the method further comprises reformulating Eq. 6, one obtains the following implicit adsorption isotherm expression: wherein γ₁ and γ_{ϕ} are functions of x₁ and a relationship between the thermodynamic adsorption equilibrium constant K° and the apparent adsorption equilibrium constant K is shown in Eq. 8.

$$K ( {x}_{1} ) = K° \frac{{γ}_{ϕ} ( {x}_{1} )}{{γ}_{1} ( {x}_{1} )} . \qquad ( 8 )$$

In another aspect, the method further comprises calculating one or more pure component isotherms for gases with adsorbents including silica gels, activated carbons, zeolites and metal organic frameworks. In another aspect, the first temperature is a fixed temperature. In another aspect, the first pressure is a relative pressure with a range of 0 to 0.1. In another aspect, the method further comprises calculating one or more pure component isotherms for gases with adsorbents including silica gels, activated carbons, zeolites and metal organic frameworks at one or more temperatures. In another aspect, the site activities are further calculated with an adsorption Non-Random Two-Liquid (aNRTL) activity coefficient. In another aspect, a reference state for an occupied site with adsorbed gas component 1 is at full surface coverage and a saturated adsorption state is x₁=1. In another aspect, the method further comprises substituting the species concentrations with the species activities and calculates the species activity coefficients with the adsorption Non-Random Two-Liquid activity coefficient. In another aspect, an adsorption equilibria calculated is at least one of: thermodynamically consistent; requires few adjustable model parameters; is applicable to both pure component adsorption isotherms and multicomponent adsorption isotherms; or calculates multicomponent adsorption isotherms from pure component adsorption isotherms.

In another embodiment, the present invention includes a computerized method for thermodynamic formulation of a Langmuir isotherm comprising: performing a calculation comprising:

$${n}_{i} = {n}_{i}^{0} \frac{KP}{1 + KP} \qquad ( 1 )$$

wherein nᵢ is the adsorption amount of gas component i; nᵢ⁰ is the adsorption maximum amount; P is the gas vapor pressure, and K is the apparent adsorption equilibrium constant in which adsorption and desorption rates are proportional to a concentration of vacant sites and occupied sites; and substituting the concentration of both a vacant site and an occupied site with site activities, wherein a reference state for the vacant sites is at zero surface coverage while the reference state for the occupied sites is at full surface coverage; wherein the foregoing steps are performed by one or more processors. In one aspect, the method further comprises substituting the constant K with a thermodynamic adsorption equilibrium constant K° calculated:

$$K° = \frac{{k}_{a}}{{k}_{d}} = \frac{{a}_{AS}}{P {a}_{S}} = \frac{{γ}_{1} {x}_{1}}{γϕ ( 1 - {x}_{1} ) P}$$

wherein α_{AS} is the activity of a site occupied with an adsorbed gas A, α_{S} is an activity of the vacant site, γ₁ and γ_{ϕ} are an activity coefficient of the occupied site with adsorbed gas component 1 and an activity coefficient of the vacant site, respectively.

In another embodiment, the present invention includes a system for classifying data comprising: at least one input/output interface; a data storage; one or more processors communicably coupled to the at least one input/output interface and the data storage, wherein the one or more processors perform the step of: determining adsorption isotherms for at least one of a first temperature, a first pressure, a low temperature, or a high pressure region, or both comprising:

$$K° = \frac{{k}_{a}}{{k}_{d}} = \frac{{a}_{AS}}{P {a}_{S}} = \frac{{γ}_{1} {x}_{1}}{γϕ ( 1 - {x}_{1} ) P} \qquad ( 6 )$$

wherein α_{AS} is the activity of a site occupied with an adsorbed gas A, α_{S} is an activity of the vacant site, γ₁ and γ_{ϕ} are an activity coefficient of the occupied site with adsorbed gas component 1 and an activity coefficient of the vacant site, respectively; and receiving the data from the at least one input/output interface.

In another embodiment, the present invention includes a computer program embodied on a non-transitory computer readable storage medium that is executed using one or more processors for thermodynamic formulation of a Langmuir isotherm comprising: (a) a code segment for receiving data to calculate the Langmuir isotherm; (b) a code segment for determining adsorption isotherms for at least one of a first temperature, a first pressure, a low temperature, or a high pressure region, or both comprising:

$$K° = \frac{{k}_{a}}{{k}_{d}} = \frac{{a}_{AS}}{P {a}_{S}} = \frac{{γ}_{1} {x}_{1}}{γϕ ( 1 - {x}_{1} ) P} \qquad ( 6 )$$

wherein α_{AS} is the activity of a site occupied with an adsorbed gas A, α_{S} is an activity of the vacant site, γ₁ and γ_{ϕ}, are an activity coefficient of the occupied site with adsorbed gas component 1 and an activity coefficient of the vacant site, respectively; and (c) a code segment for outputting the data from at least one input/output interface.

### BRIEF DESCRIPTION OF THE DRAWINGS

For a more complete understanding of the features and advantages of the present invention, reference is now made to the detailed description of the invention along with the accompanying figures and in which:

FIG. 1 shows the site activity coefficients as functions of adsorption extent with different τ_{1ϕ}(α=0.3): τ_{1ϕ}=−1 (dashed line), τ_{1ϕ}=−2 (dotted dashed line) and τ_{1ϕ}=−3 (solid line). Lines originating at −4.4, −1.6 and −0.4 stands for activity coefficient of occupied sites with adsorbate gas ‘1’ while lines originating at 0.0 stands for activity coefficient of vacant sites with phantom molecule ‘φ’.

FIGS. 2A and 2B show a comparison of RMS with different models: (FIG. 2A) thermodynamic Langmuir compared to Langmuir (FIG. 2B) thermodynamic Langmuir compared to Sips.

FIGS. 3A to 3D show a comparison of adsorption isotherms with different models: (FIG. 3A) CO₂/zeolite 5A [39] at 348 K (FIG. 3B) CH₄/zeolite 5A [22] at 343 K (FIG. 3C) N₂ /Zeolite 5A [22] at 343 K and (FIG. 3D) CH₄/activated carbon at 212.7 K. Experimental data ( [symbol] ), Langmuir ( [symbol] ), Sips ( [symbol] ), and Thermodynamic Langmuir ( [symbol] ).

FIGS. 4A and 4B show the

$$ln ( \frac{K}{K°} )$$

of (FIG. 4A) N₂/zeolite 5A [22] at 273 K ( [symbol] ), 303 K ( [symbol] ), and 343 K ( [symbol] ); (FIG. 4B) CH₄/activated carbon at 212.7 K ( [symbol] ), 260.2 K ( [symbol] ) and 304.1 K ( [symbol] ).

FIGS. 5A to 5C show the adsorption strength of (FIG. 5A) CH₄, (FIG. 5B) CO₂, and (FIG. 5C) N₂ in different adsorbents. silica gel ( [symbol] ), activated carbon ( [symbol] ), zeolite 5A ( [symbol] ), zeolite 13X ( [symbol] ), Cu-BTC ( [symbol] ), UiO-66 ( [symbol] ), and Zn-MOF (+).

FIGS. 6A and 6B show the adsorption isotherm of (FIG. 6A) C₃H₈, (FIG. 6B) i-C₄H₁₀ in Cu-BTC at 348 K. experimental data ( [symbol] ), Langmuir ( [symbol] ), Sips ( [symbol] ), Thermodynamic Langmuir ( [symbol] ).

FIGS. 7A and 7B show the ratio of thermodynamic adsorption equilibrium constant and observed apparent adsorption equilibrium constant for (FIG. 7A) C₃H₈ and (FIG. 7B) i-C₄H₁₀ adsorption with Cu-BTC at 348 K [25].

FIGS. 8A to 8C show the correlation results with the classical Langmuir isotherm and the Sips isotherm models: (FIG. 8A) CO₂/Activated carbon [1] at 212.7 K (FIG. 8B) CO₂/Zeolite 5A [2] at 228 K and (FIG. 8C) CO₂/Zeolite 5A [2] at 272 K. Experimental data ( [symbol] ), Langmuir model ( [symbol] ), and Sips model ( [symbol] ).

FIGS. 9A to 9C show the correlation results with the classical Langmuir isotherm, the Sips isotherm, and the thermodynamic Langmuir isotherm models: (FIG. 9A) CO₂/Activated carbon [1] at 212.7 K (FIG. 9B) CO₂/Zeolite 5A [2] at 228 K and (FIG. 9C) CO₂/Zeolite 5A [2] at 273 K. Experimental data ( [symbol] ), Langmuir model ( [symbol] ), Sips model ( [symbol] ), and thermodynamic Langmuir model ( [symbol] ).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

The classical Langmuir isotherm model [8] is considered the first scientifically sound expression for pure component adsorption isotherms:

$${n}_{i} = {n}_{i}^{0} \frac{KP}{1 + KP} \qquad ( 1 )$$

where nᵢ is the adsorption amount of gas component i; nᵢ⁰ is the adsorption maximum amount; P is the gas vapor pressure. Indicative of the affinity between adsorbate and adsorbent, K is the apparent adsorption equilibrium constant. The Langmuir isotherm has been extensively used to describe adsorption behavior of many systems including adsorption of non-polar gases on activated carbons and zeolites. Ignoring the surface heterogeneity and the van der Waals interactions between adsorbates and adsorbents [9, 10], the Langmuir isotherm may be inadequate in describing pure component adsorption isotherms especially at low temperature and high pressure regions [11] (see Example 2).

As used herein, the “relative pressure” is a measure of the pressure of a component at a given system temperature. In relation to the “relative pressure”, there is also a so-called “saturation pressure” that is the maximum possible vapor pressure for the component (or molecule) at the system temperature. For example, the saturation pressure of water at boiling point (100 deg C) is 1 bar. A “Relative” pressure is the gas pressure divided by the saturation pressure of the component at the system temperature. Often, gas adsorption takes place between relative pressure of 0 to 0.1. As used herein, the “relative” pressure has a range of 0 to 0.1.

Typically, isotherms are taken at isothermal (constant temperature) condition. In other words, the temperature is fixed. It is also possible to obtain isotherms at multiple temperatures, but most often the temperature will be a fixed temperature for the system.

### EXAMPLE 1.

### Novel Langmuir Isotherm Model

Among the many efforts [12-14] to improve upon the classical Langmuir isotherm model, the empirical Sips isotherm model [12, 13] probably is the most successful one. Following Freundlich isotherm [15, 16], Sips introduced an empirical “heterogeneity” parameter m, which is usually less than unity [17], to the Langmuir isotherm. Shown in Eq. 2, the resulting

Sips isotherm expression is much more flexible in representing adsorption isotherm data.

$${n}_{i} = {n}_{i}^{0} \frac{{( KP )}^{m}}{1 + {( KP )}^{m}} \qquad ( 2 )$$

With three adjustable parameters (nᵢ⁰, K and m), the Sips isotherm expression and other similar empirical expressions are capable of correlating pure component adsorption isotherm data much better than the Langmuir isotherm could achieve with two adjustable parameters (nᵢ⁰ and K). However, the introduction of empirical heterogeneity parameter m distorts the theoretical basis of the classical Langmuir isotherm and the physical significance of the Langmuir isotherm parameters (nᵢ⁰ and K) is lost.

Instead of pursuing empirical corrections of the classical Langmuir isotherm to address the issue of adsorbent surface heterogeneity, this work re-examines the theoretical basis of the Langmuir isotherm and proposes a thermodynamic formulation of the Langmuir isotherm. Specifically, the reformulation is based on substituting the concentrations of both the vacant sites and the occupied sites with the site activities. The reference state for the vacant sites is at zero surface coverage while the reference state for the occupied sites is at full surface coverage.

The site activities are further calculated with the adsorption Non-Random Two-Liquid (aNRTL) activity coefficient model [18]. Derived from the two fluid theory [19, 20] and the assumption that the adsorbate phase nonideality is dominated by the adsorbate-adsorbent interaction, the aNRTL model has been shown to successfully correlate and predict wide varieties of mixed-gas adsorption isotherms with a single binary interaction parameter per adsorbate-adsorbate pair.

The resulting thermodynamic Langmuir isotherm should represent a theoretically rigorous refinement of the classical Langmuir isotherm and the model parameters include nᵢ⁰, the adsorption maximum, K°, the thermodynamic adsorption equilibrium constant, and τ, the aNRTL binary interaction parameter.

The subsequent sections present the formulation of the thermodynamic Langmuir isotherm, the adsorption NRTL activity coefficient model, and the model results for 98 pure component adsorption isotherms for adsorbents including silica gels, activated carbons, zeolites and metal organic frameworks (MOFs). Also presented are the results with the classical Langmuir isotherm and the Sips isotherm. Lastly, the physical interpretation of the thermodynamic Langmuir isotherm model parameters is discussed.

Thermodynamic Langmuir Isotherm. The classical Langmuir adsorption isotherm equation is derived from reaction kinetics [21]. Suppose there is an adsorption and desorption reaction of pure gas A:

A_{(g)}+S↔AS (3)

where S is the vacant site and AS is the occupied site with gas A. When this reaction reaches chemical equilibrium state at pressure P, the rates of adsorption and desorption are the same.

kₐP[S]=k_{d}[AS] (4)

where kₐ is the rate constant of adsorption, k_{d} is the rate constant of desorption, [S] is the vacant site concentration, and [AS] is the occupied site concentration. The apparent chemical equilibrium constant, K, can be written as:

$$K = \frac{{k}_{a}}{{k}_{d}} = \frac{[ AS ]}{P [ S ]} = \frac{{n}_{1}}{( {n}_{1}^{0} - {n}_{1} ) P} = \frac{{x}_{1}}{( 1 - {x}_{1} ) P} \qquad ( 5 )$$

where n₁ stands for the adsorption amount of adsorbed gas component 1, n₁⁰ stands for the adsorption maximum, and x₁ stands for the adsorption extent, i.e., the ratio of n₁ and n₁⁰. Langmuir isotherm equation, Eq. 1, can be obtained after solving for x₁. Note that here gas A and gas component 1 are denoted interchangeably.

The Langmuir isotherm assumes the adsorption and desorption rates are proportional to the concentrations of vacant sites and occupied sites respectively. In other words, the model ignores the “heterogeneity” of the adsorption sites and the apparent chemical equilibrium constant, K, should be a function of the surface coverage, or the adsorption extent, x₁.

To account for the “heterogeneity” of the adsorption sites and to achieve a rigorous thermodynamic formulation of Langmuir isotherm, the present invention substitutes the site concentrations in Eq. 5 with the site activities, i.e., the product of site concentration and site activity coefficient. See Eq. 6.

$$K° = \frac{{k}_{a}}{{k}_{d}} = \frac{{a}_{AS}}{P {a}_{S}} = \frac{{γ}_{1} {x}_{1}}{{γ}_{ϕ} ( 1 - {x}_{1} ) P} \qquad ( 6 )$$

here K° is the thermodynamic adsorption equilibrium constant, α_{AS} is the activity of the occupied site with adsorbed gas A, α_{S} is the activity of the vacant site, γ₁ and γ_{ϕ} are the activity coefficient of the occupied site with adsorbed gas component 1 and the activity coefficient of the vacant site, respectively. The reference state for the occupied site with adsorbed gas component 1 is chosen to be at full surface coverage, i.e., saturated adsorption state with x₁=1.

The reference state for the vacant site is chosen to be at zero surface coverage, i.e., the vacant adsorption state with x₁=0. In other words, γ₁=1 at x₁=1, and γ_{ϕ=1} at x₁=0.

Reformulating Eq. 6, one obtains the following implicit adsorption isotherm expression

$${n}_{1} = {n}_{1}^{0} \frac{K° {γ}_{ϕ} P}{{γ}_{1} + K° {γ}_{ϕ} P} \qquad ( 7 )$$

here γ₁ and γ_{ϕ} are functions of x₁. The relationship between the thermodynamic adsorption equilibrium constant K° and the apparent adsorption equilibrium constant K is shown in Eq. 8.

$$K ( {x}_{1} ) = K° \frac{{γ}_{ϕ} ( {x}_{1} )}{{γ}_{1} ( {x}_{1} )} \qquad ( 8 )$$

The classical Langmuir isotherm is recovered if both the activity coefficients of the occupied sites and the vacant sites are unity. However, the surface heterogeneity suggests there are vacant sites with stronger adsorption potential and vacant sites with weaker adsorption potential. It is expected that the vacant sites with stronger adsorption potential should be occupied before the sites with weaker adsorption potential. Therefore, the activity coefficient of vacant sites should start with unity at zero surface coverage (reference state) and decline and deviate from unity as the adsorption extent increases. To the contrary, the activity coefficient of occupied sites should increase and approach unity as the adsorption proceeds to full surface coverage (reference state). In other words, the inventors found negative deviations from ideal solution behavior for both the vacant sites and the occupied sites.

The Adsorption NRTL Activity Coefficient Model. The aNRTL model activity coefficient expressions [18] for two competing adsorbate components 1 and 2 on the adsorbate phase are as follows.

$$ln {γ}_{1} = {x}_{2}^{2} [ {τ}_{12} \frac{( {G}_{12} - 1 )}{{( {x}_{2} + {x}_{1} {G}_{12} )}^{2}} ] \qquad ( 9 a )$$

$${ln γ}_{2} = {x}_{1}^{2} [ {τ}_{21} \frac{( {G}_{21} - 1 )}{{( {x}_{1} + {x}_{2} {G}_{21} )}^{2}} ] \qquad ( 9 b )$$

$$with$$

$${G}_{12} = exp ( - {ατ}_{12} ) \qquad ( 10 a )$$

$${G}_{21} = exp ( - {ατ}_{21} ) \qquad ( 10 b )$$

$$and$$

$${τ}_{12} = - {τ}_{21} = \frac{{g}_{10} - {g}_{20}}{RT} \qquad ( 11 )$$

where g₁₀ is the interaction potential between adsorbate 1 and adsorbent 0, g₂₀ is the interaction potential between adsorbate 2 and adsorbent 0, R is gas constant, T is temperature, and α is the non-randomness parameter. Following the convention of NRTL model [19], a is fixed at 0.3 in this study. τ₁₂ is the binary interaction parameter for the pair of adsorbates 1 and 2.

To apply the adsorption NRTL model, the inventors followed the concept of “competition” between two adsorbate components 1 and 2 in mixed-gas adsorption equilibria. Specifically, the inventors considered pure component adsorption equilibria as a “competition” between adsorbate component 1 and a phantom molecule ϕ. In other words, while the occupied sites are covered with adsorbate component 1, the vacant sites are “occupied” by a phantom molecule ϕ. Therefore, the adsorption NRTL model becomes

$$ln {γ}_{1} = {x}_{ϕ}^{2} [ {τ}_{1 ϕ} \frac{( {G}_{1 ϕ} - 1 )}{{( {x}_{ϕ} + {x}_{1} {G}_{1 ϕ} )}^{2}} ] \qquad ( 12 a )$$

$$ln {γ}_{ϕ} = {x}_{1}^{2} [ {τ}_{ϕ 1} \frac{( {G}_{ϕ 1} - 1 )}{{( {x}_{1} + {x}_{ϕ} {G}_{ϕ 1} )}^{2}} ] \qquad ( 12 b )$$

$$with$$

$${G}_{1 ϕ} = exp ( - {ατ}_{1 ϕ} ) \qquad ( 13 a )$$

$${G}_{ϕ 1} = exp ( - {ατ}_{ϕ 1} ) \qquad ( 13 b )$$

$$and$$

$${τ}_{1 ϕ} = - {τ}_{ϕ1} = \frac{{g}_{10} - {g}_{ϕ0}}{RT} \qquad ( 14 )$$

where x_{ϕ}=1−x₁, and g₁₀ and g_{ϕ0} are the interaction potential between component 1 and adsorbent 0 and the interaction potential between phantom molecule ϕ and adsorbent 0, respectively.

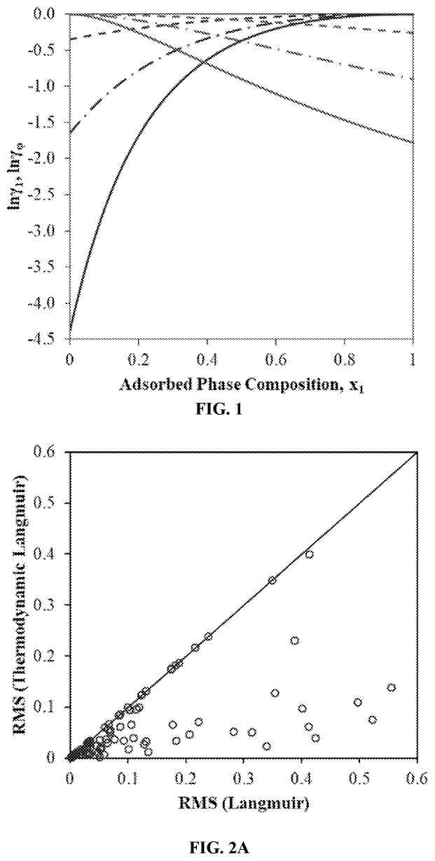

As shown later, the binary interaction parameter T_{1ϕ} is found to be in the range of 0 to −5 for the test systems of the present invention. The activity coefficients show negative deviation from ideality and the negative deviation increases as T_{1ϕ} becomes more negative, suggesting stronger attractive interaction between the adsorbate and the adsorbent (i.e., more negative g₁₀). FIG. 1 illustrates the variations in activity coefficients with the adsorption extent as τ_{1ϕ} changes. γ₁ shows negative deviation from unity in the beginning of adsorption process (weaker desorption strength) and approaches unity when the adsorption reaches saturation (reference state for the occupied sites). y_{ϕ} shows an opposite trend from that of the occupied sites. γ_{ϕ} is unity in the beginning of adsorption process (reference state for the vacant cites) and then exhibits negative deviation from unity as the adsorption extent approaches saturation (weaker adsorption strength).

The inventors examined the model performance in correlating data for 98 selected pure component adsorption isotherms with the classical Langmuir isotherm model, the semi-empirical Sips isotherm model, and the thermodynamic Langmuir model. There are two adjustable parameters (nᵢ⁰ and K) with the Langmuir isotherm, three adjustable parameters (nᵢ⁰, K and m) with the Sips isotherm, and three adjustable parameters (nᵢ⁰, K° and τ_{1ϕ}) with the thermodynamic Langmuir isotherm of the present invention.

The Maximum Likelihood Objective Function is adopted in the regression of adsorption isotherm data. Specifically, the sum of square of the ratio of the difference between calculated nᵢ and experimental nᵢ to the expected standard deviation σₑₓₚₜ (set to 0.05 and same unit as nᵢ in this disclosure) by adjusting the corresponding isotherm parameters.

Obj=Σᵢ((nᵢ^{calc}−nᵢ^{expt})/σₑₓₚₜ)² (15)

where Obj is the objective function; superscripts calc and expt stand for calculated value and experimental data, respectively.

Root mean square error (RMS) was used to evaluate the performance of the three isotherm models. The RMS is defined as following:

$$R M S = \sqrt{\frac{{\sum }_{i} {( {n}_{i}^{c a l c} - {n}_{i}^{expt} )}^{2}}{N}} \qquad ( 16 )$$

where N is the number of data points for the isotherm.

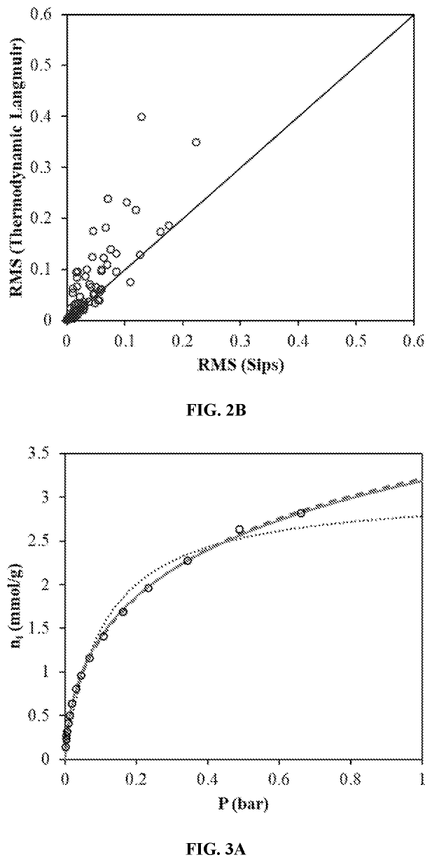

Table 1 shows the corresponding RMS values with the models. FIG. 2A and FIG. 2B show the RMS values for the isotherms with the new model plotted against those with the Langmuir isotherm and those with the Sips isotherm respectively. The results with the new model are superior to those with the Langmuir isotherm as all of the RMS data points are located in the lower right half corner of FIG. 2A. The new model is comparable to the Sips isotherm as FIG. 2B shows the RMS data points are mostly centered around the 45° line.

TABLE 1
Comparison of root mean square error among
Langmuir, Sips and Thermodynamic Langumir

| | | | | | | RMS | |
|---|---|---|---|---|---|---|---|
| System | Adsorbed | Adsorbent | | RMS | RMS | (This | Experimental |
| number | Gas | Material | T (K) | (Langmuir) | (Sips) | Study) | Data Source |
| 1 | CH₄ | Activated | 212.7 | 0.283 | 0.048 | 0.052 | Reich et al. |
| | | Carbon | 260.2 | 0.128 | 0.028 | 0.027 | [23] |
| | | | 301.4 | 0.047 | 0.022 | 0.024 | |
| 2 | CH₄ | Zeolite 5A | 273 | 0.011 | 0.009 | 0.008 | Bakhtyari |
| | | | 303 | 0.012 | 0.011 | 0.012 | and Mofarahi |
| | | | 343 | 0.005 | 0.005 | 0.005 | |
| 3 | CH₄ | Zeolite | 298 | 0.109 | 0.054 | 0.040 | Cavenati |
| | | 13X | 308 | 0.093 | 0.049 | 0.034 | et al. |
| | | | 323 | 0.036 | 0.029 | 0.029 | [27] |
| 4 | CH₄ | UiO-66 | 273 | 0.003 | 0.002 | 0.003 | Zhang et al. |
| | | | 298 | 0.001 | 0.001 | 0.001 | [28] |
| | | | 323 | 0.004 | 0.003 | 0.003 | |
| 5 | CH₄ | Zn-MOF | 273 | 0.114 | 0.019 | 0.096 | Mu and |
| | | | 282 | 0.102 | 0.016 | 0.094 | Walton |
| | | | 298 | 0.084 | 0.018 | 0.084 | |
| 6 | C₂H₄ | Silica Gel | 273.15 | 0.031 | 0.009 | 0.009 | Lewis et al. |
| | | | 298.15 | 0.009 | 0.007 | 0.007 | [29] |
| | | | 313.15 | 0.009 | 0.003 | 0.006 | |
| 7 | C₂H₄ | Zeolite 5A | 283 | 0.119 | 0.034 | 0.100 | Mofarahi and |
| | | | 303 | 0.054 | 0.023 | 0.021 | Salehi |
| | | | 323 | 0.053 | 0.014 | 0.017 | |
| 8 | C₂H₆ | Silica Gel | 278 | 0.062 | 0.029 | 0.037 | Olivier and |
| | | | 293 | 0.051 | 0.038 | 0.036 | Jadot |
| | | | 303 | 0.034 | 0.020 | 0.034 | |
| 9 | C₂H₆ | Zeolite 5A | 283 | 0.060 | 0.057 | 0.060 | Mofarahi and |
| | | | 303 | 0.029 | 0.029 | 0.029 | Salehi |
| | | | 323 | 0.023 | 0.018 | 0.018 | |
| 10 | C₃H₆ | Silica Gel | 273.15 | 0.077 | 0.038 | 0.037 | Lewis et al. |
| | | | 298.15 | 0.066 | 0.056 | 0.056 | |
| | | | 313.15 | 0.051 | 0.011 | 0.003 | |
| 11 | C₃H₆ | Activated | 303.15 | 0.412 | 0.060 | 0.061 | Laukhuf and |
| | | Carbon | 313.15 | 0.354 | 0.127 | 0.128 | Plank |
| | | | 323.15 | 0.314 | 0.047 | 0.051 | [30] |
| 12 | C₃H₆ | Zeolite | 323 | 0.086 | 0.010 | 0.062 | Campo et al. |
| | | 13X | 373 | 0.177 | 0.042 | 0.066 | [31, 32] |
| | | | 423 | 0.101 | 0.019 | 0.018 | |
| 13 | C₃H₆ | Cu-BTC | 323 | 0.349 | 0.223 | 0.349 | Ferreira et al. |
| | | | 348 | 0.131 | 0.085 | 0.131 | [25, 33] |
| | | | 373 | 0.124 | 0.044 | 0.124 | |
| 14 | C₃H₈ | Silica Gel | 273.15 | 0.064 | 0.013 | 0.030 | Lewis et al. |
| | | | 298.15 | 0.030 | 0.011 | 0.019 | [29, 34] |
| | | | 313.15 | 0.017 | 0.010 | 0.012 | |
| 15 | C₃H₈ | Activated | 293.15 | 0.413 | 0.129 | 0.399 | Payne et al. |
| | | Carbon | 303.15 | 0.497 | 0.069 | 0.110 | [35] |
| | | | 313.15 | 0.401 | 0.060 | 0.097 | |
| 16 | C₃H₈ | Zeolite | 323 | 0.034 | 0.017 | 0.020 | Campo et al. |
| | | 13X | 373 | 0.070 | 0.047 | 0.050 | [31, 32] |
| | | | 423 | 0.033 | 0.025 | 0.033 | |
| 17 | C₃H₈ | Cu-BTC | 323 | 0.216 | 0.119 | 0.216 | Ferreira et al. |
| | | | 348 | 0.123 | 0.064 | 0.123 | [25] |
| | | | 373 | 0.086 | 0.032 | 0.086 | |
| 18 | i-C4H10 | Zeolite | 298.15 | 0.106 | 0.050 | 0.066 | Hyun and |
| | | 13X | 323.15 | 0.053 | 0.028 | 0.020 | Danner |
| | | | 373.15 | 0.031 | 0.031 | 0.031 | [36] |
| 19 | i-C4H10 | Cu-BTC | 323 | 0.239 | 0.071 | 0.239 | Ferreira et al. |
| | | | 348 | 0.182 | 0.067 | 0.182 | [25, 33] |
| | | | 373 | 0.175 | 0.045 | 0.175 | |
| 20 | C₅H₁₂ | Activated | 333 | 0.222 | 0.039 | 0.071 | Do and Do |
| | | Carbon | 353 | 0.206 | 0.022 | 0.046 | [37] |
| | | | 423 | 0.135 | 0.011 | 0.012 | |
| 21 | C₅H₁₂ | Zeolite 5A | 373 | 0.033 | 0.006 | 0.006 | Silva and |
| | | | 423 | 0.034 | 0.006 | 0.009 | Rodrigues |
| | | | 473 | 0.059 | 0.006 | 0.006 | [38] |
| 22 | CO₂ | Silica Gel | 283.15 | 0.008 | 0.003 | 0.005 | Wang and |
| | | | 298.15 | 0.005 | 0.002 | 0.004 | LeVan |
| | | | 313.15 | 0.003 | 0.001 | 0.002 | [39] |
| 23 | CO₂ | Activated | 273.15 | 0.069 | 0.010 | 0.054 | Zhang et al. |
| | | Carbon | 298.15 | 0.029 | 0.007 | 0.024 | [24] |
| | | | 348.15 | 0.008 | 0.004 | 0.007 | |
| 24 | CO₂ | Zeolite 5A | 228.15 | 0.425 | 0.056 | 0.040 | Wang and |
| | | | 273.15 | 0.339 | 0.030 | 0.023 | LeVan |
| | | | 323.15 | 0.183 | 0.019 | 0.034 | [39] |
| | | | 348.15 | 0.131 | 0.015 | 0.033 | |
| 25 | CO₂ | Zeolite | 298 | 0.555 | 0.076 | 0.139 | Cavenati |
| | | 13X | 308 | 0.523 | 0.110 | 0.075 | et al. |
| | | | 323 | 0.388 | 0.103 | 0.231 | [27] |
| 26 | CO₂ | Cu-BTC | 293.15 | 0.100 | 0.060 | 0.100 | Al-Janabi |
| | | | 333.15 | 0.067 | 0.018 | 0.067 | et al. [26] |
| 27 | CO₂ | UiO-66 | 273 | 0.019 | 0.012 | 0.018 | Zhang et al. |
| | | | 298 | 0.012 | 0.010 | 0.012 | [28] |
| | | | 323 | 0.008 | 0.008 | 0.008 | |
| 28 | CO₂ | Zn-MOF | 273 | 0.188 | 0.176 | 0.186 | Mu and |
| | | | 282 | 0.175 | 0.162 | 0.174 | Walton |
| | | | 298 | 0.103 | 0.085 | 0.095 | |
| 29 | N₂ | Activated | 298.15 | 0.002 | 0.002 | 0.002 | Maring and |
| | | Carbon | 323.15 | 0.001 | 0.001 | 0.001 | Webley |
| | | | 348.15 | 0.001 | 0.001 | 0.001 | [40] |
| 30 | N₂ | Zeolite 5A | 273 | 0.017 | 0.005 | 0.008 | Bakhtyari |
| | | | 303 | 0.007 | 0.007 | 0.007 | and Mofarahi |
| | | | 343 | 0.004 | 0.004 | 0.004 | [22] |
| 31 | N₂ | Zeolite | 298 | 0.049 | 0.019 | 0.011 | Cavenati |
| | | 13X | 308 | 0.034 | 0.015 | 0.008 | et al. |
| | | | 323 | 0.027 | 0.013 | 0.009 | [27] |
| 32 | N₂ | Cu-BTC | 293.15 | 0.006 | 0.006 | 0.006 | Al-Janabi et al. |
| | | | 333.15 | 0.008 | 0.008 | 0.008 | [26] |
| 33 | N₂ | UiO-66 | 273 | 0.002 | 0.002 | 0.002 | Zhang et al. |
| | | | 298 | 0.001 | 0.001 | 0.001 | [28] |
| | | | 323 | 0.001 | 0.001 | 0.001 | |

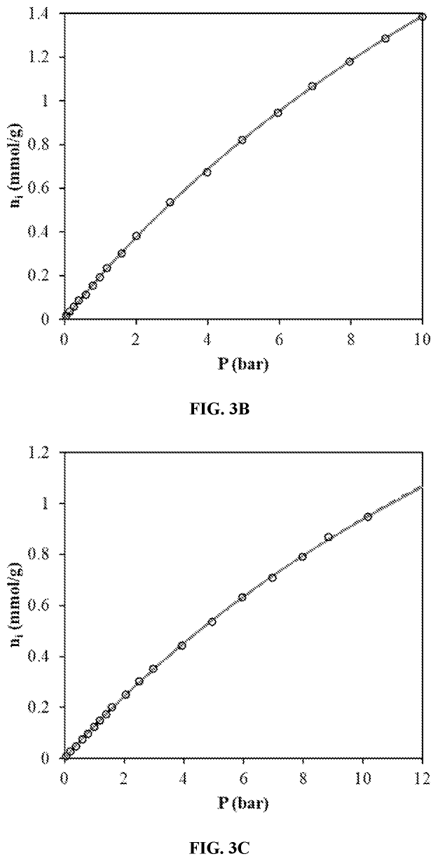

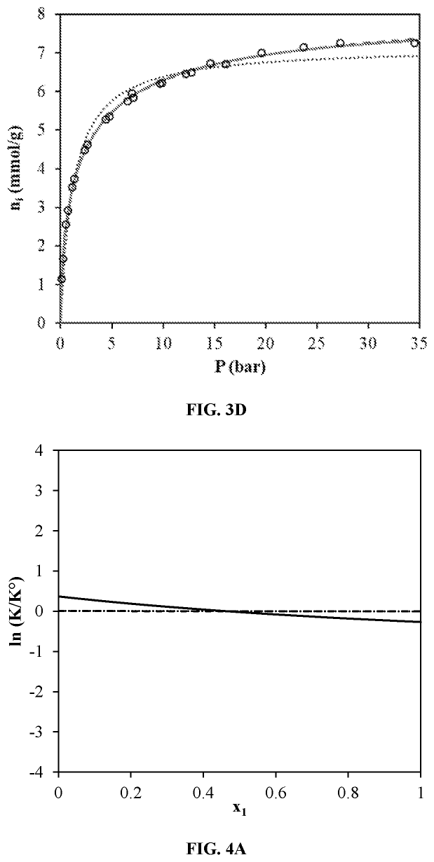

FIGS. 3A to 3C present the adsorption isotherm model results for CO₂, CH₄ and N₂ in zeolite 5A, respectively. FIG. 3A shows the Langmuir isotherm fails to accurately describe the CO₂-zeolite 5A isotherm at 348 K while the Sips isotherm and the new model fit the experimental data very well. All three models are able to fit the experimental data accurately for CH₄ and N₂ adsorption isotherms with zeolite 5A [22], as shown in FIGS. 3B and 3C respectively. FIG. 3D further shows the Langmuir isotherm fails to describe the CH₄ adsorption isotherm with activated carbon [23] while the isotherm is well represented with both the Sips isotherm and the thermodynamic Langmuir isotherm.

Tables 2 to 4 report the regressed model parameters for Langmuir, Sips and the new model respectively. From the regressed parameters for Langmuir and for Sips, it becomes obvious that the Langmuir nᵢ⁰ and K parameters can be altered significantly when the “heterogeneity” parameter m is introduced in the Sips isotherm. The changes are particularly pronounced when m is far from unity. Take CO₂ adsorption with activated carbon (AC-800-1) [24] as an example, with m≈0.8, the Sips nᵢ⁰ values are 5 to 10 times of the Langmuir nᵢ⁰ values while the Sips K values are one order of magnitude less than that of the Langmuir K values.

TABLE 2
Regressed Parameters for Langmuir Isotherm

| System | Adsorbed | Adsorbent | | | |
|---|---|---|---|---|---|
| Number | Gas | Material | T (K) | nᵢ⁰ (mmol/g) | K (bar⁻¹) |
| 1 | CH₄ | Activated | 212.7 | 7.170 ± 0.606 | 0.813 ± 0.465 |
| | | Carbon | 260.2 | 6.110 ± 0.023 | 0.230 ± 0.003 |
| | | | 301.4 | 5.217 ± 0.037 | 0.112 ± 0.002 |
| 2 | CH₄ | Zeolite 5A | 273 | 3.170 ± 0.027 | 0.398 ± 0.010 |
| | | | 303 | 3.187 ± 0.088 | 0.175 ± 0.011 |
| | | | 343 | 4.286 ± 0.581 | 0.048 ± 0.009 |
| 3 | CH₄ | Zeolite | 298 | 6.935 ± 0.039 | 0.078 ± 0.014 |
| | | 13X | 308 | 6.583 ± 0.043 | 0.068 ± 0.014 |
| | | | 323 | 6.323 ± 0.062 | 0.056 ± 0.020 |
| 4 | CH₄ | UiO-66 | 273 | 4.664 ± 0.166 | 0.241 ± 0.010 |
| | | | 298 | 3.306 ± 0.223 | 0.194 ± 0.015 |
| | | | 323 | 1.863 ± 0.231 | 0.268 ± 0.040 |
| 5 | CH₄ | Zn-MOF | 273 | 10.569 ± 0.142 | 0.055 ± 0.001 |
| | | | 282 | 10.403 ± 0.157 | 0.047 ± 0.001 |
| | | | 298 | 10.061 ± 0.192 | 0.040 ± 0.001 |
| 6 | C₂H₄ | Silica Gel | 273.15 | 2.272 ± 0.089 | 2.050 ± 0.171 |
| | | | 298.15 | 2.056 ± 0.218 | 0.851 ± 0.142 |
| | | | 313.15 | 1.578 ± 0.257 | 0.797 ± 0.200 |
| 7 | C₂H₄ | Zeolite 5A | 283 | 3.052 ± 0.014 | 13.704 ± 0.180 |
| | | | 303 | 2.832 ± 0.016 | 7.629 ± 0.273 |
| | | | 323 | 2.577 ± 0.013 | 7.497 ± 0.091 |
| 8 | C₂H₆ | Silica Gel | 278 | 6.625 ± 0.100 | 0.099 ± 0.003 |
| | | | 293 | 6.437 ± 0.182 | 0.068 ± 0.004 |
| | | | 303 | 5.508 ± 0.144 | 0.063 ± 0.003 |
| 9 | C₂H₆ | Zeolite 5A | 283 | 2.407 ± 0.016 | 6.089 ± 0.240 |
| | | | 303 | 2.253 ± 0.016 | 3.586 ± 0.109 |
| | | | 323 | 2.105 ± 0.020 | 2.257 ± 0.091 |
| 10 | C₃H₆ | Silica Gel | 273.15 | 3.660 ± 0.033 | 5.142 ± 0.722 |
| | | | 298.15 | 2.791 ± 0.051 | 3.098 ± 0.144 |
| | | | 313.15 | 2.313 ± 0.070 | 2.874 ± 0.216 |
| 11 | C₃H₆ | Activated | 303.15 | 8.675 ± 0.025 | 12.307 ± 0.306 |
| | | Carbon | 313.15 | 8.097 ± 0.016 | 12.284 ± 0.122 |
| | | | 323.15 | 8.007 ± 0.007 | 9.104 ± 0.079 |
| 12 | C₃H₆ | Zeolite | 323 | 3.272 ± 0.012 | 141.798 ± 4.894 |
| | | 13X | 373 | 3.063 ± 0.012 | 33.717 ± 0.938 |
| | | | 423 | 2.733 ± 0.018 | 7.230 ± 0.213 |
| 13 | C₃H₆ | Cu-BTC | 323 | 3.227 ± 0.015 | 11.212 ± 0.132 |
| | | | 348 | 3.113 ± 0.028 | 6.805 ± 0.454 |
| | | | 373 | 3.334 ± 0.052 | 2.557 ± 0.103 |
| 14 | C₃H₈ | Silica Gel | 273.15 | 3.935 ± 0.156 | 1.713 ± 0.138 |
| | | | 298.15 | 2.895 ± 0.813 | 1.080 ± 0.552 |
| | | | 313.15 | 2.415 ± 0.229 | 0.813 ± 0.118 |
| 15 | C₃H₈ | Activated | 293.15 | 6.446 ± 0.042 | 10.717 ± 0.032 |
| | | Carbon | 303.15 | 6.233 ± 0.013 | 8.329 ± 0.098 |
| | | | 313.15 | 6.286 ± 0.199 | 5.534 ± 2.010 |
| 16 | C₃H₈ | Zeolite | 323 | 3.015 ± 0.036 | 18.826 ± 4.833 |
| | | 13X | 373 | 2.786 ± 0.018 | 5.138 ± 0.176 |
| | | | 423 | 2.725 ± 0.036 | 1.226 ± 0.069 |
| 17 | C₃H₈ | Cu-BTC | 323 | 2.880 ± 0.447 | 6.563 ± 2.267 |
| | | | 348 | 2.839 ± 0.044 | 3.001 ± 0.074 |
| | | | 373 | 2.904 ± 0.038 | 1.454 ± 0.110 |
| 18 | i-C₄H₁₀ | Zeolite | 298.15 | 1.723 ± 0.194 | 380.995 ± 81.356 |
| | | 13X | 323.15 | 1.553 ± 0.013 | 153.869 ± 9.134 |
| | | | 373.15 | 1.373 ± 0.092 | 28.305 ± 1.605 |
| 19 | i-C₄H₁₀ | Cu-BTC | 323 | 2.358 ± 0.016 | 25.807 ± 0.888 |
| | | | 348 | 2.276 ± 0.020 | 18.921 ± 0.731 |
| | | | 373 | 2.358 ± 0.028 | 6.666 ± 0.292 |
| 20 | C₅H₁₂ | Activated | 333 | 3.267 ± 0.019 | 523.338 ± 10.552 |
| | | Carbon | 353 | 3.159 ± 0.022 | 230.454 ± 9.028 |
| | | | 423 | 2.533 ± 0.020 | 32.713 ± 0.924 |
| 21 | C₅H₁₂ | Zeolite 5A | 373 | 1.259 ± 0.021 | 120.398 ± 13.734 |
| | | | 423 | 1.051 ± 0.019 | 59.272 ± 5.925 |
| | | | 473 | 0.933 ± 0.020 | 21.813 ± 2.015 |
| 22 | CO₂ | Silica Gel | 283.15 | 3.060 ± 0.453 | 0.829 ± 0.185 |
| | | | 298.15 | 2.897 ± 0.633 | 0.533 ± 0.160 |
| | | | 313.15 | 2.398 ± 1.312 | 0.425 ± 0.294 |
| 23 | CO₂ | Activated | 273.15 | 13.112 ± 0.434 | 0.617 ± 0.029 |
| | | Carbon | 298.15 | 9.385 ± 0.672 | 0.459 ± 0.044 |
| | | | 348.15 | 5.762 ± 1.836 | 0.252 ± 0.096 |
| 24 | CO₂ | Zeolite 5A | 228.15 | 4.389 ± 0.024 | 3035.631 ± 154.139 |
| | | | 273.15 | 4.201 ± 0.059 | 149.269 ± 5.356 |
| | | | 323.15 | 3.474 ± 0.199 | 19.563 ± 1.298 |
| | | | 348.15 | 3.086 ± 0.028 | 9.241 ± 0.206 |
| 25 | CO₂ | Zeolite | 298 | 6.826 ± 0.014 | 3.361 ± 0.041 |
| | | 13X | 308 | 6.206 ± 0.009 | 2.958 ± 0.015 |
| | | | 323 | 5.190 ± 0.017 | 1.971 ± 0.021 |
| 26 | CO₂ | Cu-BTC | 293.15 | 15.549 ± 0.019 | 0.484 ± 0.003 |
| | | | 333.15 | 15.200 ± 0.047 | 0.142 ± 0.001 |
| 27 | CO₂ | UiO-66 | 273 | 8.196 ± 0.297 | 0.562 ± 0.029 |
| | | | 298 | 6.153 ± 0.825 | 0.345 ± 0.058 |
| | | | 323 | 4.616 ± 1.908 | 0.221 ± 0.106 |
| 28 | CO₂ | Zn-MOF | 273 | 14.982 ± 0.041 | 0.157 ± 0.001 |
| | | | 282 | 15.171 ± 0.056 | 0.115 ± 0.001 |
| | | | 298 | 15.669 ± 0.085 | 0.075 ± 0.001 |
| 29 | N₂ | Activated | 298.15 | 0.225 ± 0.041 | 1.681 ± 0.614 |
| | | Carbon | 323.15 | 0.172 ± 0.047 | 1.486 ± 0.785 |
| | | | 348.15 | 0.171 ± 0.075 | 1.009 ± 0.737 |
| 30 | N₂ | Zeolite 5A | 273 | 2.463 ± 0.058 | 0.256 ± 0.013 |
| | | | 303 | 2.800 ± 0.168 | 0.103 ± 0.010 |
| | | | 343 | 3.359 ± 0.740 | 0.039 ± 0.011 |
| 31 | N₂ | Zeolite | 298 | 6.197 ± 0.072 | 0.042 ± 0.001 |
| | | 13X | 308 | 6.199 ± 0.082 | 0.034 ± 0.001 |
| | | | 323 | 5.978 ± 0.113 | 0.028 ± 0.001 |
| 32 | N₂ | Cu-BTC | 293.15 | 15.787 ± 0.751 | 0.018 ± 0.001 |
| | | | 333.15 | 8.987 ± 0.750 | 0.019 ± 0.002 |
| 33 | N₂ | UiO-66 | 273 | 2.193 ± 0.114 | 0.117 ± 0.007 |
| | | | 298 | 1.616 ± 0.103 | 0.086 ± 0.006 |
| | | | 323 | 1.437 ± 0.775 | 0.027 ± 0.009 |

TABLE 3
Regressed Parameters for Sips Isotherm

| System | Adsorbed | Adsorbent | | | | |
|---|---|---|---|---|---|---|
| number | Gas | Material | T (K) | nᵢ⁰ (mmol/g) | K (bar⁻¹) | m |
| 1 | CH₄ | Activated | 212.7 | 8.595 ± 0.040 | 0.481 ± 0.006 | 0.635 ± 0.006 |
| | | Carbon | 260.2 | 7.127 ± 0.091 | 0.154 ± 0.006 | 0.765 ± 0.013 |
| | | | 301.4 | 5.762 ± 0.123 | 0.088 ± 0.005 | 0.879 ± 0.020 |
| 2 | CH₄ | Zeolite 5A | 273 | 3.233 ± 0.110 | 0.379 ± 0.032 | 0.975 ± 0.038 |
| | | | 303 | 3.117 ± 0.235 | 0.184 ± 0.029 | 1.017 ± 0.052 |
| | | | 343 | 4.800 ± 2.224 | 0.040 ± 0.029 | 0.975 ± 0.089 |
| 3 | CH₄ | Zeolite | 298 | 8.756 ± 0.218 | 0.043 ± 0.003 | 0.800 ± 0.014 |
| | | 13X | | | | |
| | | | 308 | 8.306 ± 0.227 | 0.038 ± 0.003 | 0.820 ± 0.014 |
| | | | 323 | 6.944 ± 0.230 | 0.046 ± 0.003 | 0.932 ± 0.020 |
| 4 | CH₄ | UiO-66 | 273 | 6.024 ± 0.972 | 0.168 ± 0.037 | 0.969 ± 0.016 |
| | | | 298 | 3.289 ± 0.146 | 0.196 ± 0.014 | 1.000 ± 0.012 |
| | | | 323 | 2.506 ± 0.358 | 0.169 ± 0.035 | 0.947 ± 0.003 |
| 5 | CH₄ | Zn-MOF | 273 | 21.149 ± 2.020 | 0.013 ± 0.003 | 0.751 ± 0.017 |
| | | | 282 | 22.583 ± 3.162 | 0.010 ± 0.003 | 0.762 ± 0.020 |
| | | | 298 | 23.822 ± 4.944 | 0.008 ± 0.003 | 0.770 ± 0.025 |
| 6 | C₂H₄ | Silica Gel | 273.15 | 3.635 ± 0.796 | 0.702 ± 0.350 | 0.775 ± 0.060 |
| | | | 298.15 | 2.678 ± 0.145 | 0.519 ± 0.053 | 0.909 ± 0.014 |
| | | | 313.15 | 3.067 ± 0.407 | 0.239 ± 0.055 | 0.840 ± 0.018 |
| 7 | C₂H₄ | Zeolite 5A | 283 | 3.313 ± 0.023 | 14.053 ± 0.079 | 0.617 ± 0.020 |
| | | | 303 | 2.931 ± 0.047 | 7.761 ± 0.375 | 0.820 ± 0.004 |
| | | | 323 | 2.681 ± 0.005 | 7.264 ± 0.006 | 0.805 ± 0.001 |
| 8 | C₂H₆ | Silica Gel | 278 | 8.320 ± 0.047 | 0.059 ± 0.002 | 0.839 ± 0.026 |
| | | | 293 | 7.131 ± 0.012 | 0.055 ± 0.001 | 0.928 ± 0.005 |
| | | | 303 | 7.022 ± 0.172 | 0.038 ± 0.002 | 0.873 ± 0.651 |
| 9 | C₂H₆ | Zeolite 5A | 283 | 2.375 ± 0.008 | 6.124 ± 0.022 | 1.094 ± 0.005 |
| | | | 303 | 2.264 ± 0.053 | 3.561 ± 0.023 | 0.976 ± 0.054 |
| | | | 323 | 2.149 ± 0.001 | 2.151 ± 0.011 | 0.926 ± 0.002 |
| 10 | C₃H₆ | Silica Gel | 273.15 | 4.959 ± 0.556 | 2.251 ± 1.628 | 0.686 ± 0.574 |
| | | | 298.15 | 3.699 ± 0.292 | 1.552 ± 0.164 | 0.776 ± 0.663 |
| | | | 313.15 | 5.383 ± 2.724 | 0.321 ± 0.433 | 0.621 ± 0.086 |
| 11 | C₃H₆ | Activated | 303.15 | 14.317 ± 0.342 | 2.042 ± 0.244 | 0.463 ± 0.344 |
| | | Carbon | 313.15 | 11.473 ± 0.181 | 4.010 ± 0.216 | 0.556 ± 0.013 |
| | | | 323.15 | 12.003 ± 0.251 | 2.537 ± 0.205 | 0.547 ± 0.010 |
| 12 | C₃H₆ | Zeolite | 323 | 3.783 ± 0.468 | 840.010 ± 214.551 | 0.278 ± 0.123 |
| | | 13X | 373 | 3.510 ± 0.048 | 24.199 ± 1.484 | 0.494 ± 0.020 |
| | | | 423 | 3.020 ± 0.027 | 5.156 ± 0.124 | 0.710 ± 0.018 |
| 13 | C₃H₆ | Cu-BTC | 323 | 2.933 ± 0.016 | 15.017 ± 0.265 | 2.402 ± 0.057 |
| | | | 348 | 2.909 ± 0.021 | 7.860 ± 0.196 | 1.393 ± 0.045 |
| | | | 373 | 2.609 ± 0.044 | 4.138 ± 0.131 | 1.665 ± 0.076 |
| 14 | C₃H₈ | Silica Gel | 273.15 | 9.772 ± 3.128 | 0.232 ± 0.155 | 0.707 ± 0.033 |
| | | | 298.15 | 7.818 ± 4.749 | 0.159 ± 0.177 | 0.765 ± 0.060 |
| | | | 313.15 | 5.205 ± 4.474 | 0.203 ± 0.303 | 0.824 ± 0.102 |
| 15 | C₃H₈ | Activated | 293.15 | 7.794 ± 0.037 | 5.166 ± 0.077 | 0.566 ± 0.007 |
| | | Carbon | 303.15 | 8.127 ± 0.091 | 3.056 ± 0.173 | 0.474 ± 0.008 |
| | | | 313.15 | 7.694 ± 0.039 | 2.621 ± 0.032 | 0.526 ± 0.006 |
| 16 | C₃H₈ | Zeolite | 323 | 3.082 ± 0.031 | 18.192 ± 1.076 | 0.844 ± 0.050 |
| | | 13X | 373 | 2.956 ± 0.037 | 4.369 ± 0.184 | 0.824 ± 0.027 |
| | | | 423 | 2.583 ± 0.053 | 1.388 ± 0.069 | 1.105 ± 0.039 |
| 17 | C₃H₈ | Cu-BTC | 323 | 2.532 ± 0.019 | 9.144 ± 0.196 | 2.100 ± 0.081 |
| | | | 348 | 2.517 ± 0.017 | 3.840 ± 0.085 | 1.597 ± 0.021 |
| | | | 373 | 2.467 ± 0.045 | 2.132 ± 0.100 | 1.456 ± 0.070 |
| 18 | i-C₄H₁₀ | Zeolite | 298.15 | 2.056 ± 0.074 | 223.777 ± 44.366 | 0.396 ± 0.038 |
| | | 13X | 323.15 | 1.643 ± 0.027 | 156.647 ± 7.083 | 0.660 ± 0.039 |
| | | | 373.15 | 1.359 ± 0.031 | 29.104 ± 2.244 | 1.038 ± 0.072 |
| 19 | 1-C₄H₁₀ | Cu-BTC | 323 | 2.169 ± 0.013 | 27.611 ± 0.400 | 3.656 ± 0.133 |
| | | | 348 | 2.068 ± 0.016 | 22.271 ± 0.499 | 2.255 ± 0.122 |
| | | | 373 | 1.991 ± 0.018 | 9.050 ± 0.194 | 2.558 ± 0.160 |
| 20 | C₅H₁₂ | Activated | 333 | 4.132 ± 0.036 | 214.488 ± 10.536 | 0.432 ± 0.014 |
| | | Carbon | 353 | 4.232 ± 0.058 | 74.941 ± 4.783 | 0.419 ± 0.014 |
| | | | 423 | 4.413 ± 0.535 | 4.474 ± 2.315 | 0.475 ± 0.038 |
| 21 | C₅H₁₂ | Zeolite 5A | 373 | 1.457 ± 0.180 | 142.658 ± 47.690 | 0.472 ± 0.185 |
| | | | 423 | 1.335 ± 0.194 | 36.870 ± 22.510 | 0.455 ± 0.132 |
| | | | 473 | 0.998 ± 0.073 | 18.889 ± 3.755 | 0.830 ± 0.134 |
| 22 | CO₂ | Silica Gel | 283.15 | 4.269 ± 0.296 | 0.456 ± 0.555 | 0.905 ± 0.141 |
| | | | 298.15 | 4.294 ± 0.503 | 0.278 ± 0.522 | 0.918 ± 0.173 |
| | | | 313.15 | 3.805 ± 1.181 | 0.211 ± 0.955 | 0.932 ± 0.314 |
| 23 | CO₂ | Activated | 273.15 | 75.786 ± 6.820 | 0.035 ± 0.005 | 0.782 ± 0.003 |
| | | Carbon | 298.15 | 29.977 ± 2.880 | 0.075 ± 0.011 | 0.852 ± 0.005 |
| | | | 348.15 | 20.837 ± 8.120 | 0.044 ± 0.022 | 0.903 ± 0.012 |
| 24 | CO₂ | Zeolite 5A | 228.15 | 6.596 ± 0.558 | 552.472 ± 200.118 | 0.434 ± 0.044 |
| | | | 273.15 | 6.265 ± 0.155 | 28.816 ± 3.434 | 0.481 ± 0.010 |
| | | | 323.15 | 6.126 ± 0.177 | 2.991 ± 0.205 | 0.552 ± 0.009 |
| | | | 348.15 | 6.059 ± 0.654 | 1.231 ± 0.413 | 0.585 ± 0.024 |
| 25 | CO₂ | Zeolite | 298 | 8.822 ± 0.094 | 1.266 ± 0.080 | 0.422 ± 0.007 |
| | | 13X | 308 | 8.647 ± 0.045 | 0.672 ± 0.027 | 0.413 ± 0.022 |
| | | | 323 | 6.986 ± 0.062 | 0.586 ± 0.005 | 0.482 ± 0.019 |
| 26 | CO₂ | Cu-BTC | 293.15 | 15.179 ± 0.030 | 0.512 ± 0.003 | 1.067 ± 0.005 |
| | | | 333.15 | 14.303 ± 0.095 | 0.163 ± 0.002 | 1.080 ± 0.009 |
| 27 | CO₂ | UiO-66 | 273 | 11.624 ± 3.744 | 0.311 ± 0.166 | 0.917 ± 0.056 |
| | | | 298 | 11.544 ± 1.409 | 0.134 ± 0.238 | 0.913 ± 0.109 |
| | | | 323 | 4.788 ± 0.948 | 0.210 ± 0.066 | 0.995 ± 0.045 |
| 28 | CO₂ | Zn-MOF | 273 | 15.746 ± 0.176 | 0.140 ± 0.122 | 0.936 ± 0.008 |
| | | | 282 | 16.255 ± 0.162 | 0.098 ± 0.182 | 0.926 ± 0.010 |
| | | | 298 | 17.352 ± 0.085 | 0.060 ± 0.345 | 0.922 ± 0.003 |
| 29 | N₂ | Activated | 298.15 | 0.218 ± 0.012 | 1.801 ± 0.198 | 1.027 ± 0.044 |
| | | Carbon | 323.15 | 0.176 ± 0.016 | 1.419 ± 0.273 | 0.984 ± 0.061 |
| | | | 348.15 | 0.153 ± 0.019 | 1.245 ± 0.283 | 1.065 ± 0.076 |
| 30 | N₂ | Zeolite 5A | 273 | 2.854 ± 0.267 | 0.182 ± 0.040 | 0.883 ± 0.057 |
| | | | 303 | 2.811 ± 0.533 | 0.102 ± 0.037 | 0.998 ± 0.082 |
| | | | 343 | 3.545 ± 2.641 | 0.036 ± 0.040 | 0.990 ± 0.130 |
| 31 | N₂ | Zeolite | 298 | 7.812 ± 0.363 | 0.025 ± 0.003 | 0.857 ± 0.020 |
| | | 13X | 308 | 7.549 ± 0.380 | 0.022 ± 0.002 | 0.895 ± 0.020 |
| | | | 323 | 7.410 ± 0.563 | 0.018 ± 0.003 | 0.906 ± 0.025 |
| 32 | N₂ | Cu-BTC | 293.15 | 16.368 ± 2.465 | 0.017 ± 0.004 | 0.993 ± 0.029 |
| | | | 333.15 | 9.732 ± 2.940 | 0.017 ± 0.008 | 0.985 ± 0.053 |
| 33 | N₂ | UiO-66 | 273 | 2.575 ± 0.368 | 0.096 ± 0.017 | 0.989 ± 0.008 |
| | | | 298 | 1.978 ± 0.260 | 0.066 ± 0.011 | 0.985 ± 0.007 |
| | | | 323 | 1.784 ± 0.752 | 0.039 ± 0.018 | 1.017 ± 0.009 |

TABLE 4
Regressed Parameters for Thermodynamic Langmuir Isotherm

| System | Adsorbed | Adsorbent | | | | |
|---|---|---|---|---|---|---|
| number | Gas | Material | T (K) | ni0 (mmol/g) | K° (bar⁻¹) | τ_{1φ} |
| 1 | CH₄ | Activated | 212.7 | 7.958 ± 0.036 | 0.686 ± 0.011 | −1.887 ± 0.029 |
| | | Carbon | 260.2 | 6.818 ± 0.265 | 0.182 ± 0.046 | −1.369 ± 0.033 |
| | | | 301.4 | 5.697 ± 0.063 | 0.092 ± 0.024 | −0.962 ± 0.025 |
| 2 | CH₄ | Zeolite 5A | 273 | 3.247 ± 0.102 | 0.376 ± 0.029 | −0.486 ± 0.282 |
| | | | 303 | 3.187 ± 0.051 | 0.175 ± 0.006 | −0.011 ± 0.005 |
| | | | 343 | 6.056 ± 2.443 | 0.026 ± 0.019 | −0.892 ± 0.488 |
| 3 | CH₄ | Zeolite | 298 | 9.246 ± 0.339 | 0.040 ± 0.004 | −1.445 ± 0.066 |
| | | 13X | 308 | 9.055 ± 0.288 | 0.033 ± 0.002 | −1.414 ± 0.051 |
| | | | 323 | 7.216 ± 0.619 | 0.042 ± 0.019 | −0.854 ± 0.026 |
| 4 | CH₄ | UiO-66 | 273 | 5.913 ± 0.681 | 0.164 ± 0.025 | −0.678 ± 0.179 |
| | | | 298 | 4.127 ± 0.838 | 0.137 ± 0.249 | −0.639 ± 0.159 |
| | | | 323 | 2.410 ± 0.666 | 0.173 ± 0.171 | −0.763 ± 0.216 |
| 5 | CH₄ | Zn-MOF | 273 | 11.462 ± 0.370 | 0.046 ± 0.024 | −0.732 ± 0.084 |
| | | | 282 | 10.878 ± 1.435 | 0.043 ± 0.017 | −0.586 ± 0.022 |
| | | | 298 | 9.966 ± 1.802 | 0.041 ± 0.032 | −0.199 ± 0.035 |
| 6 | C₂H₄ | Silica Gel | 273.15 | 7.623 ± 1.030 | 0.122 ± 0.044 | −2.140 ± 0.106 |
| | | | 298.15 | 5.479 ± 4.708 | 0.122 ± 0.229 | −1.654 ± 0.707 |
| | | | 313.15 | 6.356 ± 3.449 | 0.045 ± 0.060 | −1.970 ± 0.425 |
| 7 | C₂H₄ | Zeolite 5A | 283 | 3.236 ± 0.334 | 10.500 ± 1.164 | −1.571 ± 0.237 |
| | | | 303 | 2.894 ± 0.197 | 8.356 ± 0.941 | −1.320 ± 0.148 |
| | | | 323 | 2.639 ± 0.412 | 7.853 ± 1.471 | −1.337 ± 0.299 |
| 8 | C₂H₆ | Silica Gel | 278 | 9.557 ± 0.623 | 0.045 ± 0.008 | −1.389 ± 0.073 |
| | | | 293 | 8.412 ± 0.774 | 0.040 ± 0.041 | −1.054 ± 0.728 |
| | | | 303 | 5.539 ± 0.068 | 0.062 ± 0.005 | −0.206 ± 0.009 |
| 9 | C₂H₆ | Zeolite 5A | 283 | 2.408 ± 0.003 | 6.090 ± 0.045 | −0.180 ± 0.053 |
| | | | 303 | 2.258 ± 0.029 | 3.579 ± 0.020 | −0.327 ± 0.038 |
| | | | 323 | 2.134 ± 0.064 | 2.200 ± 0.017 | −0.718 ± 0.031 |
| 10 | C₃H₆ | Silica Gel | 273.15 | 4.589 ± 0.046 | 3.055 ± 0.165 | −1.610 ± 0.071 |
| | | | 298.15 | 3.746 ± 0.334 | 1.596 ± 0.142 | −1.432 ± 0.084 |
| | | | 313.15 | 3.631 ± 0.700 | 1.010 ± 0.189 | −1.458 ± 0.500 |
| 11 | C₃H₆ | Activated | 303.15 | 11.109 ± 0.269 | 7.346 ± 0.105 | −2.452 ± 0.015 |
| | | Carbon | 313.15 | 9.853 ± 0.148 | 8.030 ± 0.064 | −2.086 ± 0.122 |
| | | | 323.15 | 10.130 ± 0.079 | 5.489 ± 0.189 | −2.113 ± 0.071 |
| 12 | C₃H₆ | Zeolite | 323 | 3.311 ± 0.036 | 5425.52 ± 6235.90 | −5.277 ± 0.751 |
| | | 13X | 373 | 3.231 ± 0.059 | 51.594 ± 4.683 | −2.927 ± 0.159 |
| | | | 423 | 2.892 ± 0.126 | 6.261 ± 0.159 | −1.618 ± 0.109 |
| 13 | C₃H₆ | Cu-BTC | 323 | 3.227 ± 0.015 | 11.212 ± 0.132 | 0 |
| | | | 348 | 3.113 ± 0.028 | 6.805 ± 0.454 | 0 |
| | | | 373 | 3.334 ± 0.052 | 2.557 ± 0.103 | 0 |
| 14 | C₃H₈ | Silica Gel | 273.15 | 13.404 ± 3.714 | 0.104 ± 0.076 | −2.108 ± 0.217 |
| | | | 298.15 | 10.864 ± 5.704 | 0.063 ± 0.083 | −2.025 ± 0.418 |
| | | | 313.15 | 9.764 ± 1.068 | 0.044 ± 0.012 | −1.988 ± 0.087 |
| 15 | C₃H₈ | Activated | 293.15 | 7.927 ± 0.139 | 10.582 ± 0.097 | −3.603 ± 0.156 |
| | | Carbon | 303.15 | 7.008 ± 0.220 | 7.070 ± 0.550 | −2.697 ± 0.215 |
| | | | 313.15 | 6.907 ± 0.336 | 4.786 ± 0.429 | −2.376 ± 0.183 |
| 16 | C₃H₈ | Zeolite | 323 | 3.054 ± 0.013 | 19.014 ± 1.006 | −1.246 ± 0.173 |
| | | 13X | 373 | 2.891 ± 0.070 | 4.753 ± 0.035 | −1.172 ± 0.012 |
| | | | 423 | 2.725 ± 0.036 | 1.226 ± 0.069 | 0 |
| 17 | C₃H₈ | Cu-BTC | 323 | 2.880 ± 0.447 | 6.563 ± 2.267 | 0 |
| | | | 348 | 2.839 ± 0.044 | 3.001 ± 0.074 | 0 |
| | | | 373 | 2.904 ± 0.038 | 1.454 ± 0.110 | 0 |
| 18 | i-C₄H₁₀ | Zeolite | 298.15 | 1.862 ± 0.040 | 3342.87 ± 337.05 | −5.268 ± 1.007 |
| | | 13X | 323.15 | 1.606 ± 0.016 | 211.566 ± 41.245 | −2.328 ± 0.3534 |
| | | | 373.15 | 1.373 ± 0.092 | 28.305 ± 1.605 | 0 |
| 19 | i-C₄H₁₀ | Cu-BTC | 323 | 2.358 ± 0.016 | 25.807 ± 0.888 | 0 |
| | | | 348 | 2.276 ± 0.020 | 18.921 ± 0.731 | 0 |
| | | | 373 | 2.358 ± 0.028 | 6.666 ± 0.292 | 0 |
| 20 | C₅H₁₂ | Activated | 333 | 3.638 ± 0.043 | 727.284 ± 95.591 | −3.406 ± 0.207 |
| | | Carbon | 353 | 3.587 ± 0.290 | 290.096 ± 59.780 | −3.301 ± 0.459 |
| | | | 423 | 3.464 ± 0.236 | 14.819 ± 3.259 | −2.395 ± 0.144 |
| 21 | C₅H₁₂ | Zeolite 5A | 373 | 1.321 ± 0.044 | 2025.04 ± 1000.35 | −4.897 ± 0.255 |
| | | | 423 | 1.157 ± 0.071 | 141.843 ± 107.771 | −3.327 ± 1.135 |
| | | | 473 | 0.972 ± 0.044 | 20.729 ± 2.493 | −1.111 ± 0.506 |
| 22 | CO₂ | Silica Gel | 283.15 | 4.356 ± 0.418 | 0.431 ± 0.082 | −1.010 ± 0.131 |
| | | | 298.15 | 4.191 ± 0.048 | 0.277 ± 0.022 | −0.954 ± 0.008 |
| | | | 313.15 | 3.408 ± 0.057 | 0.234 ± 0.032 | −0.873 ± 0.034 |
| 23 | CO₂ | Activated | 273.15 | 34.030 ± 5.725 | 0.099 ± 0.028 | −1.575 ± 0.146 |
| | | Carbon | 298.15 | 16.865 ± 3.339 | 0.161 ± 0.575 | −1.181 ± 0.819 |
| | | | 348.15 | 8.719 ± 2.535 | 0.127 ± 0.039 | −0.903 ± 0.283 |
| 24 | CO₂ | Zeolite 5A | 228.15 | 5.368 ± 0.061 | 1909.55 ± 178.01 | −2.719 ± 0.119 |
| | | | 273.15 | 5.242 ± 0.228 | 77.102 ± 13.378 | −2.519 ± 0.080 |
| | | | 323.15 | 5.874 ± 0.520 | 4.366 ± 1.192 | −2.314 ± 0.090 |
| | | | 348.15 | 5.460 ± 0.621 | 2.045 ± 0.494 | −2.107 ± 0.043 |
| 25 | CO₂ | Zeolite | 298 | 7.514 ± 0.414 | 3.785 ± 0.639 | −2.987 ± 0.373 |
| | | 13X | 308 | 6.334 ± 0.241 | 3.599 ± 0.172 | −2.496 ± 0.229 |
| | | | 323 | 5.920 ± 0.307 | 0.990 ± 0.139 | −2.090 ± 0.480 |
| 26 | CO₂ | Cu-BTC | 293.15 | 15.549 ± 0.019 | 0.484 ± 0.003 | 0 |
| | | | 333.15 | 15.200 ± 0.047 | 0.142 ± 0.001 | 0 |
| 27 | CO₂ | UiO-66 | 273 | 9.109 ± 0.700 | 0.465 ± 0.197 | −0.552 ± 0.071 |
| | | | 298 | 8.547 ± 1.819 | 0.198 ± 0.098 | −0.838 ± 0.095 |
| | | | 323 | 5.570 ± 0.467 | 0.163 ± 0.205 | −0.602 ± 0.050 |
| 28 | CO₂ | Zn-MOF | 273 | 15.352 ± 0.095 | 0.149 ± 0.029 | −0.517 ± 0.066 |
| | | | 282 | 15.796 ± 0.156 | 0.105 ± 0.003 | −0.595 ± 0.070 |
| | | | 298 | 17.109 ± 0.401 | 0.062 ± 0.003 | −0.724 ± 0.086 |
| 29 | N₂ | Activated | 298.15 | 0.226 ± 0.123 | 1.664 ± 0.024 | −0.163 ± 0.044 |
| | | Carbon | 323.15 | 0.180 ± 0.015 | 1.362 ± 0.116 | −0.466 ± 0.044 |
| | | | 348.15 | 0.171 ± 0.042 | 1.008 ± 0.057 | −0.003 ± 0.047 |
| 30 | N₂ | Zeolite 5A | 273 | 2.909 ± 0.345 | 0.177 ± 0.047 | −1.022 ± 0.302 |
| | | | 303 | 2.808 ± 0.005 | 0.102 ± 0.028 | −0.108 ± 0.024 |
| | | | 343 | 3.291 ± 1.226 | 0.040 ± 0.021 | −0.154 ± 0.079 |
| 31 | N₂ | Zeolite | 298 | 9.405 ± 0.993 | 0.017 ± 0.002 | −1.388 ± 0.277 |
| | | 13X | 308 | 9.424 ± 0.055 | 0.014 ± 0.001 | −1.288 ± 0.002 |
| | | | 323 | 9.441 ± 0.059 | 0.011 ± 0.002 | −1.254 ± 0.012 |
| 32 | N₂ | Cu-BTC | 293.15 | 15.739 ± 0.261 | 0.018 ± 0.003 | −0.081 ± 0.003 |
| | | | 333.15 | 8.987 ± 0.750 | 0.019 ± 0.002 | 0 |
| 33 | N₂ | UiO-66 | 273 | 2.193 ± 0.114 | 0.117 ± 0.007 | 0 |
| | | | 298 | 1.616 ± 0.103 | 0.086 ± 0.006 | 0 |
| | | | 323 | 1.437 ± 0.775 | 0.027 ± 0.009 | 0 |

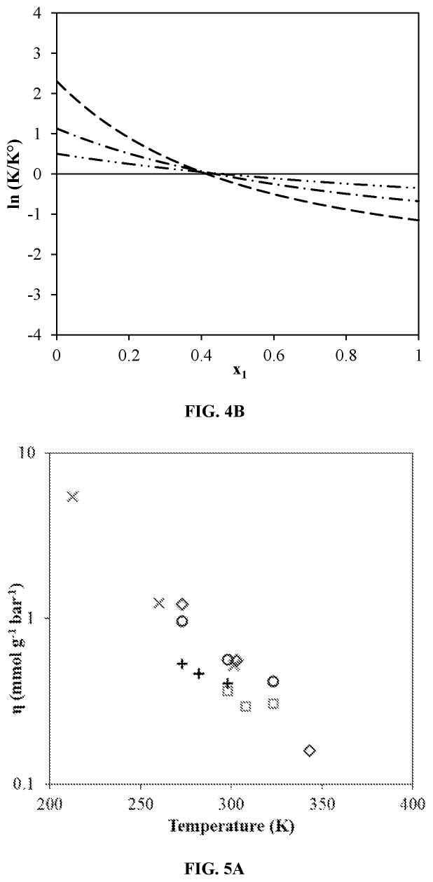

By contrast, the thermodynamic Langmuir nᵢ⁰ and K° remain in line with the Langmuir nᵢ⁰ and K. In fact, the thermodynamic Langmuir K° is an intrinsic quantity and it is related to the Langmuir K with Eq. 8. FIGS. 4A and 4B show comparisons of the thermodynamic Langmuir In K° and the Langmuir ln K for N₂ adsorption with zeolite 5A [22] and CH₄ adsorption with activated carbon respectively. While the thermodynamic Langmuir ln K° remains constant at a given temperature, the Langmuir ln K decreases with the adsorption extent. It is worth noting that the τ_{1ϕ} is near zero for the N₂/zeolite 5A system, the Langmuir ln K deviates only slightly from the thermodynamic Langmuir ln K°, and the classical Langmuir should be able to capture the isotherm data well. To the contrary, the absolute value of τ_{1ϕ} is significantly larger for the CH₄/activated carbon system, the Langmuir ln K deviates significantly from the thermodynamic Langmuir ln K°, and the classical Langmuir would fail to describe the adsorption isotherm.

Given the thermodynamic Langmuir nᵢ⁰ and K°, one may define a thermodynamic driving force for adsorption, or adsorption strength η, as the product of nᵢ⁰ and K°.

η=nᵢ⁰k° (16)

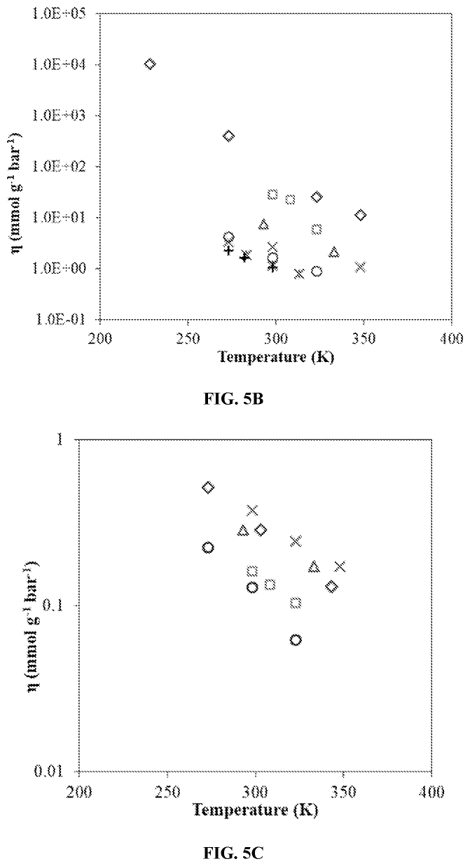

FIGS. 5A to 5C show the adsorption strength for CH₄, CO₂ and N₂ in various adsorbents respectively. The adsorption strength declines as temperature increases. η could be an effective measure to select adsorbents for a given separation task since the unit of η is adsorption amount per adsorbent unit mass per unit pressure. In other words, η has the same unit as the Henry's constant H. The relation between the Henry's constant H and the adsorption strength η can be obtained from Eq. 7 when pressure is approaching zero:

$$H = \frac{η}{{γ}_{1} ( P \to 0 )} = \frac{η}{{γ}_{1}^{∞}} \qquad ( 17 )$$

where y₁^{∞} is the infinite dilution activity coefficient and always less than or equal to unity. Different from the Henry's constant, the adsorption strength η evaluates the adsorption strength of the entire isotherm instead of considering only the low pressure region. Given η, for example, zeolite (FIG. 5A) is the strongest of the adsorbents shown in FIG. 5B for CO₂ adsorption.

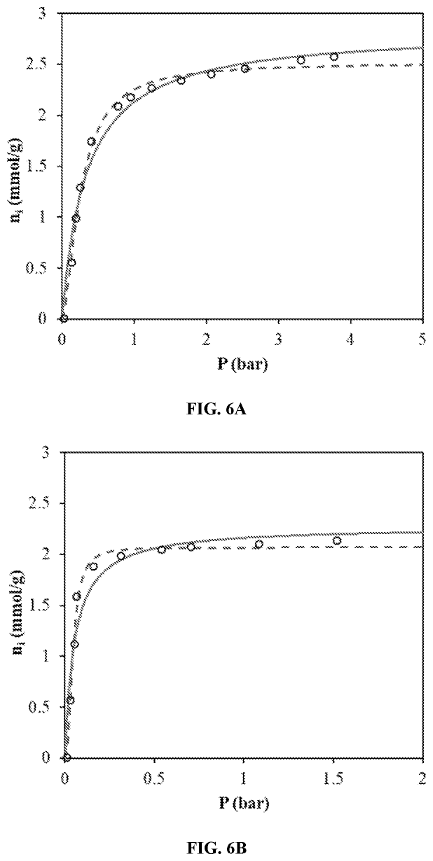

While the new model is successful in capturing adsorption behavior of most systems, Table 1 shows that the thermodynamic Langmuir is not able to capture well the experimental data for systems with Cu-BTC MOF [25, 26]. The identified T_{1ϕ}'s for these systems are all around zero, suggesting ideal solution behavior. Sips isotherm is able to correlate the data slightly better, albeit with Sips parameter m greater than unity. FIGS. 6A and 6B present the isotherms for C₃H₈ and i-C₄H₁₀ adsorption with Cu-BTC [25] respectively. These isotherms show near step change behavior in reaching saturation. For these systems, thermodynamic Langmuir initially overpredicts and then underpredicts nᵢ at the low adsorption region, and it predicts relatively well at the high adsorption region. To the contrary, Sips predicts well at the low adsorption region but underpredicts at the high adsorption region.

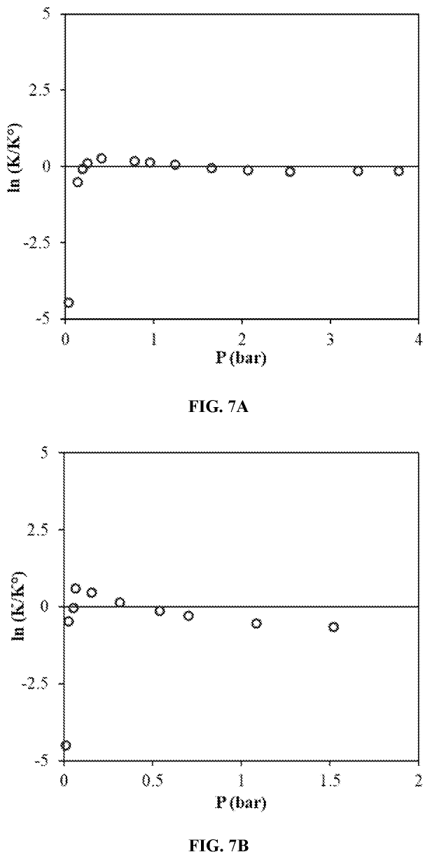

FIGS. 7A and 7B show the ratio of the observed apparent adsorption equilibrium constant to the thermodynamic adsorption equilibrium constant, ln K/K°, calculated from the isotherm data for C₃H₈ and i-C₄H₁₀ systems in Cu-BTC [25] respectively. For both systems, the observed ln K/K° values jump in the beginning of adsorption and then quickly reach a constant value of 0 as pressure increases. By way of explanation, and in no way a limitation of the present invention, is that the adsorption data points at low pressure (<0.1 bar) may be subject to higher relative uncertainty although the literature did not report the corresponding uncertainty. If the first adsorption data point at very low pressure is removed, the thermodynamic Langmuir clearly captures the isotherm data of Cu-BTC systems very well.

A thermodynamic Langmuir isotherm model is demonstrated by introducing the concept of activity and activity coefficient to the classical Langmuir isotherm. With three physically meaningful parameters, i.e., adsorption maximum amount nᵢ⁰, thermodynamic adsorption equilibrium constant K°, and binary interaction parameter τ_{1ϕ}, the model accurately describes the 98 isotherms of 33 tested adsorption systems. Based on these three parameters, further demonstrated an adsorption strength, the product of nᵢ⁰ and K°, as a measure for selecting adsorbents for a given gas adsorption task. The model is superior to the classical Langmuir and accurately correlates pure component adsorption isotherms and predicts mixed-gas adsorption isotherms. Finally, this new thermodynamic Langmuir isotherm model finally allows for determining enthalpy of adsorption and multicomponent adsorption isotherms from pure component adsorption isotherms.

### EXAMPLE 2

### Difficulty in Capturing the Adsorption Behavior with the Classical Langmuir Equation Especially at Low Temperatures and High Pressures

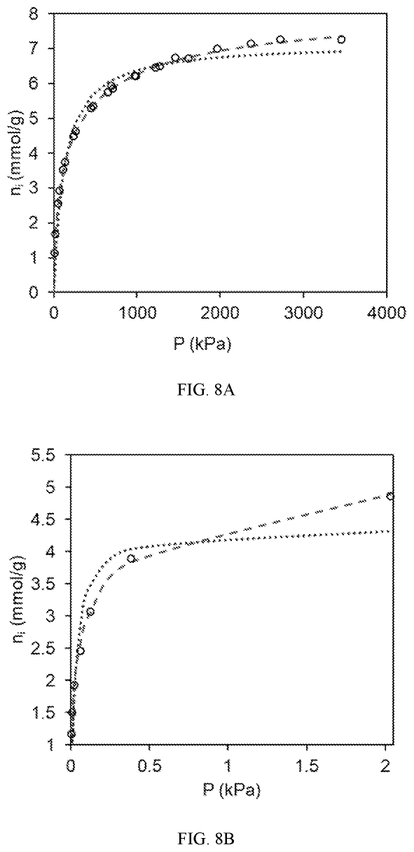

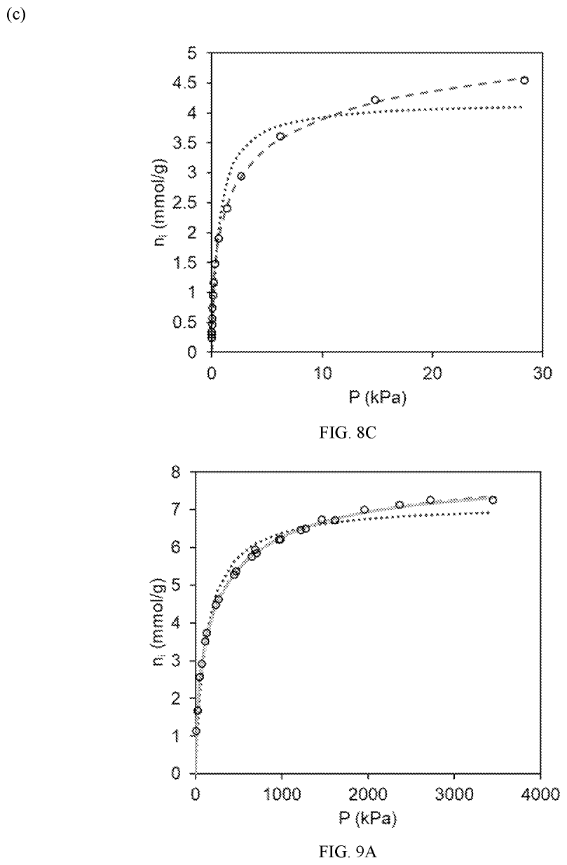

FIGS. 8A to 8C show the Langmuir isotherm captures the adsorption behavior qualitatively at low temperatures while the semi-empirical Sips model captures the experimental data quantitatively at the expense of physical significance of the Langmuir isotherm parameters.

FIGS. 8A to 8C show the correlation results with the classical Langmuir isotherm and the Sips isotherm models: (FIG. 8A) CO₂/Activated carbon [1] at 212.7 K (FIG. 8B) CO₂/Zeolite 5A [2] at 228 K and (FIG. 8C) CO₂/Zeolite 5A [2] at 272 K. Experimental data ( [symbol] ), Langmuir model ( [symbol] ), and Sips model ( [symbol] ).

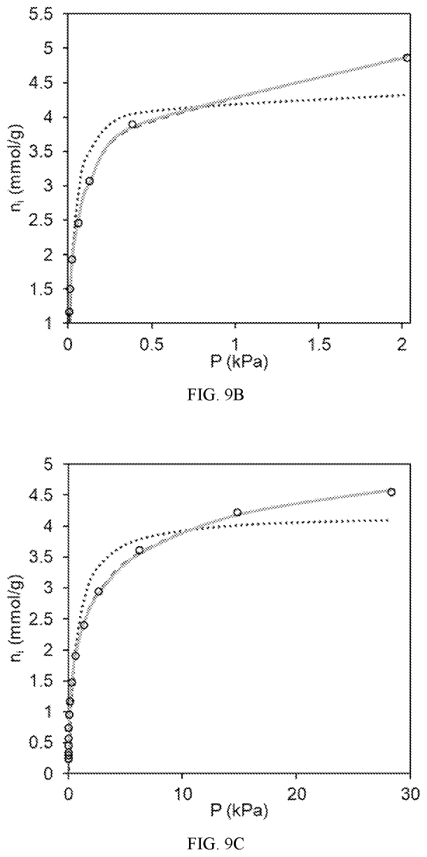

FIGS. 9A to 9C show demonstrates that the thermodynamic Langmuir is comparable to the Sips model at low temperatures while retaining physical significance of the parameters.

FIGS. 9A to 9C show the correlation results with the classical Langmuir isotherm, the Sips isotherm, and the thermodynamic Langmuir isotherm models: (FIG. 9A) CO₂/Activated carbon [1] at 212.7 K (FIG. 9B) CO₂/Zeolite 5A [2] at 228 K and (FIG. 9C) CO₂/Zeolite 5A [2] at 273 K. Experimental data ( [symbol] ), Langmuir model ( [symbol] ), Sips model ( [symbol] ), and thermodynamic Langmuir model ( [symbol] ).

TABLE 5
Regressed Parameters for Classical Langmuir Isotherm

| System | Adsorbed | Adsorbent | | | |
|---|---|---|---|---|---|
| Number | Gas | Material | T (K) | nᵢ⁰ (mmol/g) | K (bar⁻¹) |
| 1 | CH₄ | Activated | 212.7 | 7.170 ± 0.606 | 0.813 ± 0.465 |
| | | Carbon | 260.2 | 6.110 ± 0.023 | 0.230 ± 0.003 |
| | | | 301.4 | 5.217 ± 0.037 | 0.112 ± 0.002 |
| 2 | CH₄ | Zeolite 5A | 273 | 3.170 ± 0.027 | 0.398 ± 0.010 |
| | | | 303 | 3.187 ± 0.088 | 0.175 ± 0.011 |
| | | | 343 | 4.286 ± 0.581 | 0.048 ± 0.009 |
| 3 | CH₄ | Zeolite | 298 | 6.935 ± 0.039 | 0.078 ± 0.014 |
| | | 13X | 308 | 6.583 ± 0.043 | 0.068 ± 0.014 |
| | | | 323 | 6.323 ± 0.062 | 0.056 ± 0.020 |
| 4 | CH₄ | UiO-66 | 273 | 4.664 ± 0.166 | 0.241 ± 0.010 |
| | | | 298 | 3.306 ± 0.223 | 0.194 ± 0.015 |
| | | | 323 | 1.863 ± 0.231 | 0.268 ± 0.040 |
| 5 | CH₄ | Zn-MOF | 273 | 10.569 ± 0.142 | 0.055 ± 0.001 |
| | | | 282 | 10.403 ± 0.157 | 0.047 ± 0.001 |
| | | | 298 | 10.061 ± 0.192 | 0.040 ± 0.001 |
| 6 | C₂H₄ | Silica Gel | 273.15 | 2.272 ± 0.089 | 2.050 ± 0.171 |
| | | | 298.15 | 2.056 ± 0.218 | 0.851 ± 0.142 |
| | | | 313.15 | 1.578 ± 0.257 | 0.797 ± 0.200 |
| 7 | C₂H₄ | Zeolite 5A | 283 | 3.052 ± 0.014 | 13.704 ± 0.180 |
| | | | 303 | 2.832 ± 0.016 | 7.629 ± 0.273 |
| | | | 323 | 2.577 ± 0.013 | 7.497 ± 0.091 |
| 8 | C₂H₆ | Silica Gel | 278 | 6.625 ± 0.100 | 0.099 ± 0.003 |
| | | | 293 | 6.437 ± 0.182 | 0.068 ± 0.004 |
| | | | 303 | 5.508 ± 0.144 | 0.063 ± 0.003 |
| 9 | C₂H₆ | Zeolite 5A | 283 | 2.407 ± 0.016 | 6.089 ± 0.240 |
| | | | 303 | 2.253 ± 0.016 | 3.586 ± 0.109 |
| | | | 323 | 2.105 ± 0.020 | 2.257 ± 0.091 |
| 10 | C₃H₆ | Silica Gel | 273.15 | 3.660 ± 0.033 | 5.142 ± 0.722 |
| | | | 298.15 | 2.791 ± 0.051 | 3.098 ± 0.144 |
| | | | 313.15 | 2.313 ± 0.070 | 2.874 ± 0.216 |
| 11 | C₃H₆ | Activated | 303.15 | 8.675 ± 0.025 | 12.307 ± 0.306 |
| | | Carbon | 313.15 | 8.097 ± 0.016 | 12.284 ± 0.122 |
| | | | 323.15 | 8.007 ± 0.007 | 9.104 ± 0.079 |
| 12 | C₃H₆ | Zeolite | 323 | 3.272 ± 0.012 | 141.798 ± 4.894 |
| | | 13X | 373 | 3.063 ± 0.012 | 33.717 ± 0.938 |
| | | | 423 | 2.733 ± 0.018 | 7.230 ± 0.213 |
| 13 | C₃H₆ | Cu-BTC | 323 | 3.227 ± 0.015 | 11.212 ± 0.132 |
| | | | 348 | 3.113 ± 0.028 | 6.805 ± 0.454 |
| | | | 373 | 3.334 ± 0.052 | 2.557 ± 0.103 |
| 14 | C₃H₈ | Silica Gel | 273.15 | 3.935 ± 0.156 | 1.713 ± 0.138 |
| | | | 298.15 | 2.895 ± 0.813 | 1.080 ± 0.552 |
| | | | 313.15 | 2.415 ± 0.229 | 0.813 ± 0.118 |
| 15 | C₃H₈ | Activated | 293.15 | 6.446 ± 0.042 | 10.717 ± 0.032 |
| | | Carbon | 303.15 | 6.233 ± 0.013 | 8.329 ± 0.098 |
| | | | 313.15 | 6.286 ± 0.199 | 5.534 ± 2.010 |
| 16 | C₃H₈ | Zeolite | 323 | 3.015 ± 0.036 | 18.826 ± 4.833 |
| | | 13X | 373 | 2.786 ± 0.018 | 5.138 ± 0.176 |
| | | | 423 | 2.725 ± 0.036 | 1.226 ± 0.069 |
| 17 | C₃H₈ | Cu-BTC | 323 | 2.880 ± 0.447 | 6.563 ± 2.267 |
| | | | 348 | 2.839 ± 0.044 | 3.001 ± 0.074 |
| | | | 373 | 2.904 ± 0.038 | 1.454 ± 0.110 |
| 18 | i-C₄H₁₀ | Zeolite | 298.15 | 1.723 ± 0.194 | 380.995 ± 81.356 |
| | | 13X | 323.15 | 1.553 ± 0.013 | 153.869 ± 9.134 |
| | | | 373.15 | 1.373 ± 0.092 | 28.305 ± 1.605 |
| 19 | i-C₄H₁₀ | Cu-BTC | 323 | 2.358 ± 0.016 | 25.807 ± 0.888 |
| | | | 348 | 2.276 ± 0.020 | 18.921 ± 0.731 |
| | | | 373 | 2.358 ± 0.028 | 6.666 ± 0.292 |
| 20 | C₅H₁₂ | Activated | 333 | 3.267 ± 0.019 | 523.338 ± 10.552 |
| | | Carbon | 353 | 3.159 ± 0.022 | 230.454 ± 9.028 |
| | | | 423 | 2.533 ± 0.020 | 32.713 ± 0.924 |
| 21 | C₅H₁₂ | Zeolite 5A | 373 | 1.259 ± 0.021 | 120.398 ± 13.734 |
| | | | 423 | 1.051 ± 0.019 | 59.272 ± 5.925 |
| | | | 473 | 0.933 ± 0.020 | 21.813 ± 2.015 |
| 22 | CO₂ | Silica Gel | 283.15 | 3.060 ± 0.453 | 0.829 ± 0.185 |
| | | | 298.15 | 2.897 ± 0.633 | 0.533 ± 0.160 |
| | | | 313.15 | 2.398 ± 1.312 | 0.425 ± 0.294 |
| 23 | CO₂ | Activated | 273.15 | 13.112 ± 0.434 | 0.617 ± 0.029 |
| | | Carbon | 298.15 | 9.385 ± 0.672 | 0.459 ± 0.044 |
| | | | 348.15 | 5.762 ± 1.836 | 0.252 ± 0.096 |
| 24 | CO₂ | Zeolite 5A | 228.15 | 4.389 ± 0.024 | 3035.631 ± 154.139 |
| | | | 273.15 | 4.201 ± 0.059 | 149.269 ± 5.356 |
| | | | 323.15 | 3.474 ± 0.199 | 19.563 ± 1.298 |
| | | | 348.15 | 3.086 ± 0.028 | 9.241 ± 0.206 |
| 25 | CO₂ | Zeolite | 298 | 6.826 ± 0.014 | 3.361 ± 0.041 |
| | | 13X | 308 | 6.206 ± 0.009 | 2.958 ± 0.015 |
| | | | 323 | 5.190 ± 0.017 | 1.971 ± 0.021 |
| 26 | CO₂ | Cu-BTC | 293.15 | 15.549 ± 0.019 | 0.484 ± 0.003 |
| | | | 333.15 | 15.200 ± 0.047 | 0.142 ± 0.001 |
| 27 | CO₂ | UiO-66 | 273 | 8.196 ± 0.297 | 0.562 ± 0.029 |
| | | | 298 | 6.153 ± 0.825 | 0.345 ± 0.058 |
| | | | 323 | 4.616 ± 1.908 | 0.221 ± 0.106 |
| 28 | CO₂ | Zn-MOF | 273 | 14.982 ± 0.041 | 0.157 ± 0.001 |
| | | | 282 | 15.171 ± 0.056 | 0.115 ± 0.001 |
| | | | 298 | 15.669 ± 0.085 | 0.075 ± 0.001 |
| 29 | N₂ | Activated | 298.15 | 0.225 ± 0.041 | 1.681 ± 0.614 |
| | | Carbon | 323.15 | 0.172 ± 0.047 | 1.486 ± 0.785 |
| | | | 348.15 | 0.171 ± 0.075 | 1.009 ± 0.737 |
| 30 | N₂ | Zeolite 5A | 273 | 2.463 ± 0.058 | 0.256 ± 0.013 |
| | | | 303 | 2.800 ± 0.168 | 0.103 ± 0.010 |
| | | | 343 | 3.359 ± 0.740 | 0.039 ± 0.011 |
| 31 | N₂ | Zeolite | 298 | 6.197 ± 0.072 | 0.042 ± 0.001 |
| | | 13X | 308 | 6.199 ± 0.082 | 0.034 ± 0.001 |
| | | | 323 | 5.978 ± 0.113 | 0.028 ± 0.001 |
| 32 | N₂ | Cu-BTC | 293.15 | 15.787 ± 0.751 | 0.018 ± 0.001 |
| | | | 333.15 | 8.987 ± 0.750 | 0.019 ± 0.002 |
| 33 | N₂ | UiO-66 | 273 | 2.193 ± 0.114 | 0.117 ± 0.007 |
| | | | 298 | 1.616 ± 0.103 | 0.086 ± 0.006 |
| | | | 323 | 1.437 ± 0.775 | 0.027 ± 0.009 |

TABLE 6
Regressed Parameters for Sips Isotherm

| System | Adsorbed | Adsorbent | | | | |
|---|---|---|---|---|---|---|
| number | Gas | Material | T (K) | nᵢ⁰ (mmol/g) | K (bar⁻¹) | m |
| 1 | CH₄ | Activated | 212.7 | 8.595 ± 0.040 | 0.481 ± 0.006 | 0.635 ± 0.006 |
| | | Carbon | 260.2 | 7.127 ± 0.091 | 0.154 ± 0.006 | 0.765 ± 0.013 |
| | | | 301.4 | 5.762 ± 0.123 | 0.088 ± 0.005 | 0.879 ± 0.020 |
| 2 | CH₄ | Zeolite 5A | 273 | 3.233 ± 0.110 | 0.379 ± 0.032 | 0.975 ± 0.038 |
| | | | 303 | 3.117 ± 0.235 | 0.184 ± 0.029 | 1.017 ± 0.052 |
| | | | 343 | 4.800 ± 2.224 | 0.040 ± 0.029 | 0.975 ± 0.089 |
| 3 | CH₄ | Zeolite | 298 | 8.756 ± 0.218 | 0.043 ± 0.003 | 0.800 ± 0.014 |
| | | 13X | 308 | 8.306 ± 0.227 | 0.038 ± 0.003 | 0.820 ± 0.014 |
| | | | 323 | 6.944 ± 0.230 | 0.046 ± 0.003 | 0.932 ± 0.020 |
| 4 | CH₄ | UiO-66 | 273 | 6.024 ± 0.972 | 0.168 ± 0.037 | 0.969 ± 0.016 |
| | | | 298 | 3.289 ± 0.146 | 0.196 ± 0.014 | 1.000 ± 0.012 |
| | | | 323 | 2.506 ± 0.358 | 0.169 ± 0.035 | 0.947 ± 0.003 |
| 5 | CH₄ | Zn-MOF | 273 | 21.149 ± 2.020 | 0.013 ± 0.003 | 0.751 ± 0.017 |
| | | | 282 | 22.583 ± 3.162 | 0.010 ± 0.003 | 0.762 ± 0.020 |
| | | | 298 | 23.822 ± 4.944 | 0.008 ± 0.003 | 0.770 ± 0.025 |
| 6 | C₂H₄ | Silica Gel | 273.15 | 3.635 ± 0.796 | 0.702 ± 0.350 | 0.775 ± 0.060 |
| | | | 298.15 | 2.678 ± 0.145 | 0.519 ± 0.053 | 0.909 ± 0.014 |
| | | | 313.15 | 3.067 ± 0.407 | 0.239 ± 0.055 | 0.840 ± 0.018 |
| 7 | C₂H₄ | Zeolite 5A | 283 | 3.313 ± 0.023 | 14.053 ± 0.079 | 0.617 ± 0.020 |
| | | | 303 | 2.931 ± 0.047 | 7.761 ± 0.375 | 0.820 ± 0.004 |
| | | | 323 | 2.681 ± 0.005 | 7.264 ± 0.006 | 0.805 ± 0.001 |
| 8 | C₂H₆ | Silica Gel | 278 | 8.320 ± 0.047 | 0.059 ± 0.002 | 0.839 ± 0.026 |
| | | | 293 | 7.131 ± 0.012 | 0.055 ± 0.001 | 0.928 ± 0.005 |
| | | | 303 | 7.022 ± 0.172 | 0.038 ± 0.002 | 0.873 ± 0.651 |
| 9 | C₂H₆ | Zeolite 5A | 283 | 2.375 ± 0.008 | 6.124 ± 0.022 | 1.094 ± 0.005 |
| | | | 303 | 2.264 ± 0.053 | 3.561 ± 0.023 | 0.976 ± 0.054 |
| | | | 323 | 2.149 ± 0.001 | 2.151 ± 0.011 | 0.926 ± 0.002 |
| 10 | C₃H₆ | Silica Gel | 273.15 | 4.959 ± 0.556 | 2.251 ± 1.628 | 0.686 ± 0.574 |
| | | | 298.15 | 3.699 ± 0.292 | 1.552 ± 0.164 | 0.776 ± 0.663 |
| | | | 313.15 | 5.383 ± 2.724 | 0.321 ± 0.433 | 0.621 ± 0.086 |
| 11 | C₃H₆ | Activated | 303.15 | 14.317 ± 0.342 | 2.042 ± 0.244 | 0.463 ± 0.344 |
| | | Carbon | 313.15 | 11.473 ± 0.181 | 4.010 ± 0.216 | 0.556 ± 0.013 |
| | | | 323.15 | 12.003 ± 0.251 | 2.537 ± 0.205 | 0.547 ± 0.010 |
| 12 | C₃H₆ | Zeolite | 323 | 3.783 ± 0.468 | 840.010 ± 214.551 | 0.278 ± 0.123 |
| | | 13X | 373 | 3.510 ± 0.048 | 24.199 ± 1.484 | 0.494 ± 0.020 |
| | | | 423 | 3.020 ± 0.027 | 5.156 ± 0.124 | 0.710 ± 0.018 |
| 13 | C₃H₆ | Cu-BTC | 323 | 2.933 ± 0.016 | 15.017 ± 0.265 | 2.402 ± 0.057 |
| | | | 348 | 2.909 ± 0.021 | 7.860 ± 0.196 | 1.393 ± 0.045 |
| | | | 373 | 2.609 ± 0.044 | 4.138 ± 0.131 | 1.665 ± 0.076 |
| 14 | C₃H₈ | Silica Gel | 273.15 | 9.772 ± 3.128 | 0.232 ± 0.155 | 0.707 ± 0.033 |
| | | | 298.15 | 7.818 ± 4.749 | 0.159 ± 0.177 | 0.765 ± 0.060 |
| | | | 313.15 | 5.205 ± 4.474 | 0.203 ± 0.303 | 0.824 ± 0.102 |
| 15 | C₃H₈ | Activated | 293.15 | 7.794 ± 0.037 | 5.166 ± 0.077 | 0.566 ± 0.007 |
| | | Carbon | 303.15 | 8.127 ± 0.091 | 3.056 ± 0.173 | 0.474 ± 0.008 |
| | | | 313.15 | 7.694 ± 0.039 | 2.621 ± 0.032 | 0.526 ± 0.006 |
| 16 | C₃H₈ | Zeolite | 323 | 3.082 ± 0.031 | 18.192 ± 1.076 | 0.844 ± 0.050 |
| | | 13X | 373 | 2.956 ± 0.037 | 4.369 ± 0.184 | 0.824 ± 0.027 |
| | | | 423 | 2.583 ± 0.053 | 1.388 ± 0.069 | 1.105 ± 0.039 |
| 17 | C₃H₈ | Cu-BTC | 323 | 2.532 ± 0.019 | 9.144 ± 0.196 | 2.100 ± 0.081 |
| | | | 348 | 2.517 ± 0.017 | 3.840 ± 0.085 | 1.597 ± 0.021 |
| | | | 373 | 2.467 ± 0.045 | 2.132 ± 0.100 | 1.456 ± 0.070 |
| 18 | i-C₄H₁₀ | Zeolite | 298.15 | 2.056 ± 0.074 | 223.777 ± 44.366 | 0.396 ± 0.038 |
| | | 13X | 323.15 | 1.643 ± 0.027 | 156.647 ± 7.083 | 0.660 ± 0.039 |
| | | | 373.15 | 1.359 ± 0.031 | 29.104 ± 2.244 | 1.038 ± 0.072 |
| 19 | i-C₄H₁₀ | Cu-BTC | 323 | 2.169 ± 0.013 | 27.611 ± 0.400 | 3.656 ± 0.133 |
| | | | 348 | 2.068 ± 0.016 | 22.271 ± 0.499 | 2.255 ± 0.122 |
| | | | 373 | 1.991 ± 0.018 | 9.050 ± 0.194 | 2.558 ± 0.160 |
| 20 | C₅H₁₂ | Activated | 333 | 4.132 ± 0.036 | 214.488 ± 10.536 | 0.432 ± 0.014 |
| | | Carbon | 353 | 4.232 ± 0.058 | 74.941 ± 4.783 | 0.419 ± 0.014 |
| | | | 423 | 4.413 ± 0.535 | 4.474 ± 2.315 | 0.475 ± 0.038 |
| 21 | C₅H₁₂ | Zeolite 5A | 373 | 1.457 ± 0.180 | 142.658 ± 47.690 | 0.472 ± 0.185 |
| | | | 423 | 1.335 ± 0.194 | 36.870 ± 22.510 | 0.455 ± 0.132 |
| | | | 473 | 0.998 ± 0.073 | 18.889 ± 3.755 | 0.830 ± 0.134 |
| 22 | CO₂ | Silica Gel | 283.15 | 4.269 ± 0.296 | 0.456 ± 0.555 | 0.905 ± 0.141 |
| | | | 298.15 | 4.294 ± 0.503 | 0.278 ± 0.522 | 0.918 ± 0.173 |
| | | | 313.15 | 3.805 ± 1.181 | 0.211 ± 0.955 | 0.932 ± 0.314 |
| 23 | CO₂ | Activated | 273.15 | 75.786 ± 6.820 | 0.035 ± 0.005 | 0.782 ± 0.003 |
| | | Carbon | 298.15 | 29.977 ± 2.880 | 0.075 ± 0.011 | 0.852 ± 0.005 |
| | | | 348.15 | 20.837 ± 8.120 | 0.044 ± 0.022 | 0.903 ± 0.012 |
| 24 | CO₂ | Zeolite 5A | 228.15 | 6.596 ± 0.558 | 552.472 ± 200.118 | 0.434 ± 0.044 |
| | | | 273.15 | 6.265 ± 0.155 | 28.816 ± 3.434 | 0.481 ± 0.010 |
| | | | 323.15 | 6.126 ± 0.177 | 2.991 ± 0.205 | 0.552 ± 0.009 |
| | | | 348.15 | 6.059 ± 0.654 | 1.231 ± 0.413 | 0.585 ± 0.024 |
| 25 | CO₂ | Zeolite | 298 | 8.822 ± 0.094 | 1.266 ± 0.080 | 0.422 ± 0.007 |
| | | 13X | 308 | 8.647 ± 0.045 | 0.672 ± 0.027 | 0.413 ± 0.022 |
| | | | 323 | 6.986 ± 0.062 | 0.586 ± 0.005 | 0.482 ± 0.019 |
| 26 | CO₂ | Cu-BTC | 293.15 | 15.179 ± 0.030 | 0.512 ± 0.003 | 1.067 ± 0.005 |
| | | | 333.15 | 14.303 ± 0.095 | 0.163 ± 0.002 | 1.080 ± 0.009 |
| 27 | CO₂ | UiO-66 | 273 | 11.624 ± 3.744 | 0.311 ± 0.166 | 0.917 ± 0.056 |
| | | | 298 | 11.544 ± 1.409 | 0.134 ± 0.238 | 0.913 ± 0.109 |
| | | | 323 | 4.788 ± 0.948 | 0.210 ± 0.066 | 0.995 ± 0.045 |
| 28 | CO₂ | Zn-MOF | 273 | 15.746 ± 0.176 | 0.140 ± 0.122 | 0.936 ± 0.008 |
| | | | 282 | 16.255 ± 0.162 | 0.098 ± 0.182 | 0.926 ± 0.010 |
| | | | 298 | 17.352 ± 0.085 | 0.060 ± 0.345 | 0.922 ± 0.003 |
| 29 | N₂ | Activated | 298.15 | 0.218 ± 0.012 | 1.801 ± 0.198 | 1.027 ± 0.044 |
| | | Carbon | 323.15 | 0.176 ± 0.016 | 1.419 ± 0.273 | 0.984 ± 0.061 |
| | | | 348.15 | 0.153 ± 0.019 | 1.245 ± 0.283 | 1.065 ± 0.076 |
| 30 | N₂ | Zeolite 5A | 273 | 2.854 ± 0.267 | 0.182 ± 0.040 | 0.883 ± 0.057 |
| | | | 303 | 2.811 ± 0.533 | 0.102 ± 0.037 | 0.998 ± 0.082 |
| | | | 343 | 3.545 ± 2.641 | 0.036 ± 0.040 | 0.990 ± 0.130 |
| 31 | N₂ | Zeolite | 298 | 7.812 ± 0.363 | 0.025 ± 0.003 | 0.857 ± 0.020 |
| | | 13X | 308 | 7.549 ± 0.380 | 0.022 ± 0.002 | 0.895 ± 0.020 |
| | | | 323 | 7.410 ± 0.563 | 0.018 ± 0.003 | 0.906 ± 0.025 |
| 32 | N₂ | Cu-BTC | 293.15 | 16.368 ± 2.465 | 0.017 ± 0.004 | 0.993 ± 0.029 |
| | | | 333.15 | 9.732 ± 2.940 | 0.017 ± 0.008 | 0.985 ± 0.053 |
| 33 | N₂ | UiO-66 | 273 | 2.575 ± 0.368 | 0.096 ± 0.017 | 0.989 ± 0.008 |
| | | | 298 | 1.978 ± 0.260 | 0.066 ± 0.011 | 0.985 ± 0.007 |
| | | | 323 | 1.784 ± 0.752 | 0.039 ± 0.018 | 1.017 ± 0.009 |

TABLE 7
Regressed Parameters for Thermodynamic Langmuir Isotherm

| System | Adsorbed | Adsorbent | | | | |
|---|---|---|---|---|---|---|
| number | Gas | Material | T (K) | nᵢ⁰ (mmol/g) | K° (bar⁻¹) | τ_{1φ} |
| 1 | CH₄ | Activated | 212.7 | 7.958 ± 0.036 | 0.686 ± 0.011 | −1.887 ± 0.029 |
| | | Carbon | 260.2 | 6.818 ± 0.265 | 0.182 ± 0.046 | −1.369 ± 0.033 |
| | | | 301.4 | 5.697 ± 0.063 | 0.092 ± 0.024 | −0.962 ± 0.025 |
| 2 | CH₄ | Zeolite 5A | 273 | 3.247 ± 0.102 | 0.376 ± 0.029 | −0.486 ± 0.282 |
| | | | 303 | 3.187 ± 0.051 | 0.175 ± 0.006 | −0.011 ± 0.005 |
| | | | 343 | 6.056 ± 2.443 | 0.026 ± 0.019 | −0.892 ± 0.488 |
| 3 | CH₄ | Zeolite | 298 | 9.246 ± 0.339 | 0.040 ± 0.004 | −1.445 ± 0.066 |
| | | 13X | 308 | 9.055 ± 0.288 | 0.033 ± 0.002 | −1.414 ± 0.051 |
| | | | 323 | 7.216 ± 0.619 | 0.042 ± 0.019 | −0.854 ± 0.026 |
| 4 | CH₄ | UiO-66 | 273 | 5.913 ± 0.681 | 0.164 ± 0.025 | −0.678 ± 0.179 |
| | | | 298 | 4.127 ± 0.838 | 0.137 ± 0.249 | −0.639 ± 0.159 |
| | | | 323 | 2.410 ± 0.666 | 0.173 ± 0.171 | −0.763 ± 0.216 |
| 5 | CH₄ | Zn-MOF | 273 | 11.462 ± 0.370 | 0.046 ± 0.024 | −0.732 ± 0.084 |
| | | | 282 | 10.878 ± 1.435 | 0.043 ± 0.017 | −0.586 ± 0.022 |
| | | | 298 | 9.966 ± 1.802 | 0.041 ± 0.032 | −0.199 ± 0.035 |
| 6 | C₂H₄ | Silica Gel | 273.15 | 7.623 ± 1.030 | 0.122 ± 0.044 | −2.140 ± 0.106 |
| | | | 298.15 | 5.479 ± 4.708 | 0.122 ± 0.229 | −1.654 ± 0.707 |
| | | | 313.15 | 6.356 ± 3.449 | 0.045 ± 0.060 | −1.970 ± 0.425 |
| 7 | C₂H₄ | Zeolite 5A | 283 | 3.236 ± 0.334 | 10.500 ± 1.164 | −1.571 ± 0.237 |
| | | | 303 | 2.894 ± 0.197 | 8.356 ± 0.941 | −1.320 ± 0.148 |
| | | | 323 | 2.639 ± 0.412 | 7.853 ± 1.471 | −1.337 ± 0.299 |
| 8 | C₂H₆ | Silica Gel | 278 | 9.557 ± 0.623 | 0.045 ± 0.008 | −1.389 ± 0.073 |
| | | | 293 | 8.412 ± 0.774 | 0.040 ± 0.041 | −1.054 ± 0.728 |
| | | | 303 | 5.539 ± 0.068 | 0.062 ± 0.005 | −0.206 ± 0.009 |
| 9 | C₂H₆ | Zeolite 5A | 283 | 2.408 ± 0.003 | 6.090 ± 0.045 | −0.180 ± 0.053 |
| | | | 303 | 2.258 ± 0.029 | 3.579 ± 0.020 | −0.327 ± 0.038 |
| | | | 323 | 2.134 ± 0.064 | 2.200 ± 0.017 | −0.718 ± 0.031 |
| 10 | C₃H₆ | Silica Gel | 273.15 | 4.589 ± 0.046 | 3.055 ± 0.165 | −1.610 ± 0.071 |
| | | | 298.15 | 3.746 ± 0.334 | 1.596 ± 0.142 | −1.432 ± 0.084 |
| | | | 313.15 | 3.631 ± 0.700 | 1.010 ± 0.189 | −1.458 ± 0.500 |
| 11 | C₃H₆ | Activated | 303.15 | 11.109 ± 0.269 | 7.346 ± 0.105 | −2.452 ± 0.015 |
| | | Carbon | 313.15 | 9.853 ± 0.148 | 8.030 ± 0.064 | −2.086 ± 0.122 |
| | | | 323.15 | 10.130 ± 0.079 | 5.489 ± 0.189 | −2.113 ± 0.071 |
| 12 | C₃H₆ | Zeolite | 323 | 3.311 ± 0.036 | 5425.52 ± 6235.90 | −5.277 ± 0.751 |
| | | 13X | 373 | 3.231 ± 0.059 | 51.594 ± 4.683 | −2.927 ± 0.159 |
| | | | 423 | 2.892 ± 0.126 | 6.261 ± 0.159 | −1.618 ± 0.109 |
| 13 | C₃H₆ | Cu-BTC | 323 | 3.227 ± 0.015 | 11.212 ± 0.132 | 0 |
| | | | 348 | 3.113 ± 0.028 | 6.805 ± 0.454 | 0 |
| | | | 373 | 3.334 ± 0.052 | 2.557 ± 0.103 | 0 |
| 14 | C₃H₈ | Silica Gel | 273.15 | 13.404 ± 3.714 | 0.104 ± 0.076 | −2.108 ± 0.217 |
| | | | 298.15 | 10.864 ± 5.704 | 0.063 ± 0.083 | −2.025 ± 0.418 |
| | | | 313.15 | 9.764 ± 1.068 | 0.044 ± 0.012 | −1.988 ± 0.087 |
| 15 | C₃H₈ | Activated | 293.15 | 7.927 ± 0.139 | 10.582 ± 0.097 | −3.603 ± 0.156 |
| | | Carbon | 303.15 | 7.008 ± 0.220 | 7.070 ± 0.550 | −2.697 ± 0.215 |
| | | | 313.15 | 6.907 ± 0.336 | 4.786 ± 0.429 | −2.376 ± 0.183 |
| 16 | C₃H₈ | Zeolite | 323 | 3.054 ± 0.013 | 19.014 ± 1.006 | −1.246 ± 0.173 |
| | | 13X | 373 | 2.891 ± 0.070 | 4.753 ± 0.035 | −1.172 ± 0.012 |
| | | | 423 | 2.725 ± 0.036 | 1.226 ± 0.069 | 0 |
| 17 | C₃H₈ | Cu-BTC | 323 | 2.880 ± 0.447 | 6.563 ± 2.267 | 0 |
| | | | 348 | 2.839 ± 0.044 | 3.001 ± 0.074 | 0 |
| | | | 373 | 2.904 ± 0.038 | 1.454 ± 0.110 | 0 |
| 18 | i-C₄H₁₀ | Zeolite | 298.15 | 1.862 ± 0.040 | 3342.87 ± 337.05 | −5.268 ± 1.007 |
| | | 13X | 323.15 | 1.606 ± 0.016 | 211.566 ± 41.245 | −2.328 ± 0.3534 |
| | | | 373.15 | 1.373 ± 0.092 | 28.305 ± 1.605 | 0 |
| 19 | i-C₄H₁₀ | Cu-BTC | 323 | 2.358 ± 0.016 | 25.807 ± 0.888 | 0 |
| | | | 348 | 2.276 ± 0.020 | 18.921 ± 0.731 | 0 |
| | | | 373 | 2.358 ± 0.028 | 6.666 ± 0.292 | 0 |
| 20 | C₅H₁₂ | Activated | 333 | 3.638 ± 0.043 | 727.284 ± 95.591 | −3.406 ± 0.207 |
| | | Carbon | 353 | 3.587 ± 0.290 | 290.096 ± 59.780 | −3.301 ± 0.459 |
| | | | 423 | 3.464 ± 0.236 | 14.819 ± 3.259 | −2.395 ± 0.144 |
| 21 | C₅H₁₂ | Zeolite 5A | 373 | 1.321 ± 0.044 | 2025.04 ± 1000.35 | −4.897 ± 0.255 |
| | | | 423 | 1.157 ± 0.071 | 141.843 ± 107.771 | −3.327 ± 1.135 |
| | | | 473 | 0.972 ± 0.044 | 20.729 ± 2.493 | −1.111 ± 0.506 |
| 22 | CO₂ | Silica Gel | 283.15 | 4.356 ± 0.418 | 0.431 ± 0.082 | −1.010 ± 0.131 |
| | | | 298.15 | 4.191 ± 0.048 | 0.277 ± 0.022 | −0.954 ± 0.008 |
| | | | 313.15 | 3.408 ± 0.057 | 0.234 ± 0.032 | −0.873 ± 0.034 |
| 23 | CO₂ | Activated | 273.15 | 34.030 ± 5.725 | 0.099 ± 0.028 | −1.575 ± 0.146 |
| | | Carbon | 298.15 | 16.865 ± 3.339 | 0.161 ± 0.575 | −1.181 ± 0.819 |
| | | | 348.15 | 8.719 ± 2.535 | 0.127 ± 0.039 | −0.903 ± 0.283 |
| 24 | CO₂ | Zeolite 5A | 228.15 | 5.368 ± 0.061 | 1909.55 ± 178.01 | −2.719 ± 0.119 |
| | | | 273.15 | 5.242 ± 0.228 | 77.102 ± 13.378 | −2.519 ± 0.080 |
| | | | 323.15 | 5.874 ± 0.520 | 4.366 ± 1.192 | −2.314 ± 0.090 |
| | | | 348.15 | 5.460 ± 0.621 | 2.045 ± 0.494 | −2.107 ± 0.043 |
| 25 | CO₂ | Zeolite | 298 | 7.514 ± 0.414 | 3.785 ± 0.639 | −2.987 ± 0.373 |
| | | 13X | 308 | 6.334 ± 0.241 | 3.599 ± 0.172 | −2.496 ± 0.229 |
| | | | 323 | 5.920 ± 0.307 | 0.990 ± 0.139 | −2.090 ± 0.480 |
| 26 | CO₂ | Cu-BTC | 293.15 | 15.549 ± 0.019 | 0.484 ± 0.003 | 0 |
| | | | 333.15 | 15.200 ± 0.047 | 0.142 ± 0.001 | 0 |
| 27 | CO₂ | UiO-66 | 273 | 9.109 ± 0.700 | 0.465 ± 0.197 | −0.552 ± 0.071 |
| | | | 298 | 8.547 ± 1.819 | 0.198 ± 0.098 | −0.838 ± 0.095 |
| | | | 323 | 5.570 ± 0.467 | 0.163 ± 0.205 | −0.602 ± 0.050 |
| 28 | CO₂ | Zn-MOF | 273 | 15.352 ± 0.095 | 0.149 ± 0.029 | −0.517 ± 0.066 |
| | | | 282 | 15.796 ± 0.156 | 0.105 ± 0.003 | −0.595 ± 0.070 |
| | | | 298 | 17.109 ± 0.401 | 0.062 ± 0.003 | −0.724 ± 0.086 |
| 29 | N₂ | Activated | 298.15 | 0.226 ± 0.123 | 1.664 ± 0.024 | −0.163 ± 0.044 |
| | | Carbon | 323.15 | 0.180 ± 0.015 | 1.362 ± 0.116 | −0.466 ± 0.044 |
| | | | 348.15 | 0.171 ± 0.042 | 1.008 ± 0.057 | −0.003 ± 0.047 |
| 30 | N₂ | Zeolite 5A | 273 | 2.909 ± 0.345 | 0.177 ± 0.047 | −1.022 ± 0.302 |
| | | | 303 | 2.808 ± 0.005 | 0.102 ± 0.028 | −0.108 ± 0.024 |
| | | | 343 | 3.291 ± 1.226 | 0.040 ± 0.021 | −0.154 ± 0.079 |
| 31 | N₂ | Zeolite | 298 | 9.405 ± 0.993 | 0.017 ± 0.002 | −1.388 ± 0.277 |
| | | 13X | 308 | 9.424 ± 0.055 | 0.014 ± 0.001 | −1.288 ± 0.002 |
| | | | 323 | 9.441 ± 0.059 | 0.011 ± 0.002 | −1.254 ± 0.012 |
| 32 | N₂ | Cu-BTC | 293.15 | 15.739 ± 0.261 | 0.018 ± 0.003 | −0.081 ± 0.003 |
| | | | 333.15 | 8.987 ± 0.750 | 0.019 ± 0.002 | 0 |
| 33 | N₂ | UiO-66 | 273 | 2.193 ± 0.114 | 0.117 ± 0.007 | 0 |
| | | | 298 | 1.616 ± 0.103 | 0.086 ± 0.006 | 0 |
| | | | 323 | 1.437 ± 0.775 | 0.027 ± 0.009 | 0 |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±0.1, 0.5, 1, 2, 3, 4, 5, 6, 7, 10, 12 or 15%, or as understood to be within a normal tolerance in the art, for example, within 2 standard deviations of the mean. Unless otherwise clear from the context, all numerical values provided herein are modified by the term about.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES—EXAMPLE 1

[1] J.-R. Li, R. J. Kuppler, and H.-C. Zhou, “Selective gas adsorption and separation in metal-organic frameworks,” Chemical Society Reviews, vol. 38, pp. 1477-1504, 2009.

[2] A. Myers and J. M. Prausnitz, “Thermodynamics of mixed gas adsorption,” AIChE Journal, vol. 11, pp. 121-127, 1965.

[3] P. M. Mathias, R. Kumar, J. D. Moyer, J. M. Schork, S. R. Srinivasan, S. R. Auvil, et al., “Correlation of multicomponent gas adsorption by the dual-site Langmuir model. Application to nitrogen/oxygen adsorption on 5A-zeolite,” Industrial & Engineering Chemistry Research, vol. 35, pp. 2477-2483, 1996.

[4] A. L. Myers, “Prediction of adsorption of nonideal mixtures in nanoporous materials,” Adsorption, vol. 11, pp. 37-42, 2005.

[5] O. Talu and I. Zwiebel, “Multicomponent adsorption equilibria of nonideal mixtures,” AIChE Journal, vol. 32, pp. 1263-1276, 1986.

[6] K. S. Walton and D. S. Sholl, “Predicting multicomponent adsorption: 50 years of the ideal adsorbed solution theory,” AIChE Journal, vol. 61, pp. 2757-2762, 2015.

[7] S. Sircar, “Role of adsorbent heterogeneity on mixed gas adsorption,” Industrial & Engineering Chemistry Research, vol. 30, pp. 1032-1039, 1991.

[8] I. Langmuir, “The adsorption of gases on plane surfaces of glass, mica and platinum,” Journal of the American Chemical society, vol. 40, pp. 1361-1403, 1918.

[9] J. Sreńscek-Nazzal, U. Narkiewicz, A. W. Morawski, R. J. Wróbel, and B. Michalkiewicz, “Comparison of optimized isotherm models and error functions for carbon dioxide adsorption on activated carbon,” Journal of Chemical & Engineering Data, vol. 60, pp. 3148-3158, 2015.

[10] K. Foo and B. H. Hameed, “Insights into the modeling of adsorption isotherm systems,” Chemical Engineering Journal, vol. 156, pp. 2-10, 2010.

[11] P. Benard and R. Chahine, “Modeling of high-pressure adsorption isotherms above the critical temperature on microporous adsorbents: application to methane,” Langmuir, vol. 13, pp. 808-813, 1997.

[12] R. Sips, “On the structure of a catalyst surface,” The Journal of Chemical Physics, vol. 16, pp. 490-495, 1948.

[13] R. Sips, “On the structure of a catalyst surface. II,” The Journal of Chemical Physics, vol. 18, pp. 1024-1026, 1950.

[14] J. Toth, “State equation of the solid-gas interface layers,” Acta Chim. Hung., vol. 69, pp. 311-328, 1971.

[15] R. Herzog, “Kapillarchemie, eine darstellung der chemie der kolloide und verwandter gebiete. Von Dr. Herbert Freundlich. verlag der akademischen verlagsgesellschaft. Leipzig 1909. 591 Seiten. Preis 16, 30 Mk., geb. 17, 50 Mk,” Zeitschrift fur Elektrochemie und Angewandte Physikalische Chemie, vol. 15, pp. 948-948, 1909.

[16] H. R. O., “Kapillarchemie, eine Darstellung der Chemie der Kolloide und verwandter Gebiete. Von Dr. Herbert Freundlich. Verlag der Akademischen Verlagsgesellschaft. Leipzig 1909. 591 Seiten. Preis 16,30 Mk., geb. 17,50 Mk,” Zeitschrift fur Elektrochemie und angewandte physikalische Chemie, vol. 15, pp. 948-948, 1909.

[17] S. Pakseresht, M. Kazemeini, and M. M. Akbarnejad, “Equilibrium isotherms for CO, CO2, CH4 and C2H4 on the 5A molecular sieve by a simple volumetric apparatus,” Separation and Purification Technology, vol. 28, pp. 53-60, 2002.

[18] H. Kaur, H. Tun, M. Sees, and C.-C. Chen, “Local composition activity coefficient model for mixed-gas adsorption equilibria,” ed, Manuscript in Preparation, 2019.

[19] H. Renon and J. M. Prausnitz, “Local Compositions in Thermodynamic Excess Functions for Liquid Mixtures,” AIChE Journal, vol. 14, pp. 135-144, 1968.

[20] A. Ravichandran, R. Khare, and C. C. Chen, “Predicting NRTL binary interaction parameters from molecular simulations,” AIChE Journal, vol. 64, pp. 2758-2769, 2018.

[21] S. Sohn and D. Kim, “Modification of Langmuir isotherm in solution systems—definition and utilization of concentration dependent factor,” Chemosphere, vol. 58, pp. 115-123, 2005.

[22] A. Bakhtyari and M. Mofarahi, “Pure and binary adsorption equilibria of methane and nitrogen on zeolite 5A,” Journal of Chemical & Engineering Data, vol. 59, pp. 626-639, 2014.

[23] R. Reich, W. T. Ziegler, and K. A. Rogers, “Adsorption of methane, ethane, and ethylene gases and their binary and ternary mixtures and carbon dioxide on activated carbon at 212-301 K and pressures to 35 atmospheres,” Industrial & Engineering Chemistry Process Design and Development, vol. 19, pp. 336-344, 1980.

[24] Z. Zhang, J. Zhou, W. Xing, Q. Xue, Z. Yan, S. Zhuo, et al., “Critical role of small micropores in high CO2 uptake,” Physical Chemistry Chemical Physics, vol. 15, pp. 2523-2529, 2013.

[25] A. F. Ferreira, J. C. Santos, M. G. Plaza, N. Lamia, J. M. Loureiro, and A. E. Rodrigues, “Suitability of Cu-BTC extrudates for propane—propylene separation by adsorption processes,” Chemical Engineering Journal, vol. 167, pp. 1-12, 2011.

[26] N. Al-Janabi, P. Hill, L. Torrente-Murciano, A. Garforth, P. Gorgojo, F. Siperstein, et al., “Mapping the Cu-BTC metal—organic framework (HKUST-1) stability envelope in the presence of water vapour for CO2 adsorption from flue gases,” Chemical Engineering Journal, vol. 281, pp. 669-677, 2015.

[27] S. Cavenati, C. A. Grande, and A. E. Rodrigues, “Adsorption Equilibrium of Methane, Carbon Dioxide, and Nitrogen on Zeolite 13X at High Pressures,” Journal of Chemical & Engineering Data, vol. 49, pp. 1095-1101, 2004/07/01 2004.

[28] W. Zhang, H. Huang, C. Zhong, and D. Liu, “Cooperative effect of temperature and linker functionality on CO2 capture from industrial gas mixtures in metal—organic frameworks: a combined experimental and molecular simulation study,” Physical Chemistry Chemical Physics, vol. 14, pp. 2317-2325, 2012.

[29] W. Lewis, E. Gilliland, B. Chertow, and D. Bareis, “Vapor—Adsorbate equilibrium. III. the effect of temperature on the binary systems ethylene—propane, ethylene—propylene over silica gel,” Journal of the American Chemical Society, vol. 72, pp. 1160-1163, 1950.

[30] W. L. Laukhuf and C. A. Plank, “Adsorption of carbon dioxide, acetylene, ethane, and propylene on charcoal at near room temperatures,” Journal of Chemical and Engineering Data, vol. 14, pp. 48-51, 1969.

[31] M. Campo, A. Ribeiro, A. Ferreira, J. Santos, C. Lutz, J. Loureiro, et al., “New 13X zeolite for propylene/propane separation by vacuum swing adsorption,” Separation and Purification Technology, vol. 103, pp. 60-70, 2013.

[32] M. C. Campo, A. M. Ribeiro, A. Ferreira, J. C. Santos, C. Lutz, J. M. Loureiro, et al., “New 13X zeolite for propylene/propane separation by vacuum swing adsorption,” Separation and Purification Technology, vol. 103, pp. 60-70, 2013/01/15/2013.

[33] A. F. P. Ferreira, J. C. Santos, M. G. Plaza, N. Lamia, J. M. Loureiro, and A. E. Rodrigues, “Suitability of Cu-BTC extrudates for propane—propylene separation by adsorption processes,” Chemical Engineering Journal, vol. 167, pp. 1-12, 2011 Feb. 15, 2011.

[34] W. K. Lewis, E. R. Gilliland, B. Chertow, and D. Bareis, “Vapor Adsorbate Equilibrium. 3. The Effect of Temperature on the Binary Systems Ethylene-Propane, Ethylene-Propylene Over Silica Gel,” Journal of the American Chemical Society, vol. 72, pp. 1160-1163, 1950.

[35] H. Payne, G. Sturdevant, and T. Leland, “Improved two-dimensional equation of state to predict adsorption of pure and mixed hydrocarbons,” Industrial & Engineering Chemistry Fundamentals, vol. 7, pp. 363-374, 1968.

[36] S. H. Hyun and R. P. Danner, “Equilibrium adsorption of ethane, ethylene, isobutane, carbon dioxide, and their binary mixtures on 13X molecular sieves,” Journal of Chemical and Engineering Data, vol. 27, pp. 196-200, 1982.

[37] D. Do and H. Do, “Characterization of micro-mesoporous carbonaceous materials. Calculations of adsorption isotherm of hydrocarbons,” Langmuir, vol. 18, pp. 93-99, 2002.

[38] J. A. Silva and A. E. Rodrigues, “Sorption and diffusion of n-pentane in pellets of 5A zeolite,” Industrial & Engineering Chemistry Research, vol. 36, pp. 493-500, 1997.

[39] Y. Wang and M. D. LeVan, “Adsorption equilibrium of carbon dioxide and water vapor on zeolites 5A and 13X and silica gel: pure components,” Journal of Chemical & Engineering Data, vol. 54, pp. 2839-2844, 2009.

[40] B. J. Maring and P. A. Webley, “A new simplified pressure/vacuum swing adsorption model for rapid adsorbent screening for CO2 capture applications,” International Journal of Greenhouse Gas Control, vol. 15, pp. 16-31, 2013.

### REFERENCES—EXAMPLE 2

[1] R. Reich, W. T. Ziegler, and K. A. Rogers, “Adsorption of methane, ethane, and ethylene gases and their binary and ternary mixtures and carbon dioxide on activated carbon at 212-301 K and pressures to 35 atmospheres,” Industrial & Engineering Chemistry Process Design and Development, vol. 19, pp. 336-344, 1980.

[2] A. Bakhtyari and M. Mofarahi, “Pure and binary adsorption equilibria of methane and nitrogen on zeolite 5A,” Journal of Chemical & Engineering Data, vol. 59, pp. 626-639, 2014.

1. A method for thermodynamic formulation of a Langmuir isotherm comprising:
$$\text{?} - \text{?} \frac{KP}{\text{?}} \qquad ( 1 )$$
$$\text{?} \text{indicates text missing or illegible when filed}$$
where nᵢ is the adsorption amount of gas component i; nᵢ⁰ is the adsorption maximum amount; P is the gas vapor pressure, and K is the apparent adsorption equilibrium constant in which adsorption and desorption rates are proportional to a concentrations of vacant sites and occupied sites; and
substituting the concentration of both a vacant site and an occupied site with site activities, wherein a reference state for the vacant sites is at zero surface coverage while the reference state for the occupied sites is at full surface coverage.

2. The method of claim 1, further comprising substituting the constant K with a thermodynamic adsorption equilibrium constant K° calculated:
$$K \text{?} \text{?} \frac{\text{?}}{\text{?}} \text{?} \frac{\text{?}}{\text{?}} \text{?} \frac{\text{?}}{\text{?}} \qquad ( 6 )$$
$$\text{?} \text{indicates text missing or illegible when filed}$$
wherein α_{AS} is the activity of a site occupied with an adsorbed gas A, α_{S} is an activity of the vacant site, γ₁ and γ_{ϕ}are an activity coefficient of the occupied site with adsorbed gas component 1 and an activity coefficient of the vacant site, respectively.

3. The method of claim 1, wherein the reference state for a vacant site is chosen to be at zero surface coverage, wherein, γ₁=1 at x₁=1, and γ_{ϕ}1 at x₁=0.

4. The method of claim 2, further comprising reformulating Eq. 6, one obtains the following implicit adsorption isotherm expression:
$${n}_{1} = {n}_{1}^{0} \frac{\text{?}}{\text{?}} \qquad ( 7 )$$
$$\text{?} \text{indicates text missing or illegible when filed}$$
wherein γ₁ and γ_{ϕ} are functions of x₁ and a relationship between the thermodynamic adsorption equilibrium constant K° and the apparent adsorption equilibrium constant K is shown in Eq. 8.
$$K ( {x}_{1} ) - K \text{?} \frac{\text{?}}{\text{?}} . \qquad ( 8 )$$
$$\text{?} \text{indicates text missing or illegible when filed}$$

5. The method of claim 1, further comprising at least one of:
calculating one or more pure component isotherms for gases with adsorbents including silica gels, activated carbons, zeolites and metal organic frameworks;
calculating one or more pure component isotherms for gases with adsorbents including silica gels, activated carbons, zeolites and metal organic frameworks at one or more temperatures; or
substituting the species concentrations with the species activities and calculates the species activity coefficients with the adsorption Non-Random Two-Liquid activity coefficient.

6. (canceled).

7. The method of claim 1, wherein the site activities are further calculated with an adsorption Non-Random Two-Liquid (aNRTL) activity coefficient.

8. The method of claim 1, wherein a reference state for an occupied site with adsorbed gas component 1 is at full surface coverage and a saturated adsorption state is x₁=1.

9. (canceled)

10. The method of claim 1, wherein an adsorption equilibria calculated is at least one of: thermodynamically consistent; requires few adjustable model parameters; is applicable to both pure component adsorption isotherms and multicomponent adsorption isotherms; or calculates multicomponent adsorption isotherms from pure component adsorption isotherms.

11. A method of determining adsorption isotherms for at least one of: a first temperature, a first pressure, a low temperature, or a high pressure region, or both comprising:
$${n}_{1} = {n}_{1}^{0} \frac{\text{?}}{\text{?}} ; and$$
$$K \text{?} \text{?} \frac{\text{?}}{\text{?}} \text{?} \frac{\text{?}}{\text{?}} \text{?} \frac{\text{?}}{\text{?}}$$
$$\text{?} \text{indicates text missing or illegible when filed}$$
where nᵢ is the adsorption amount of gas component i; nᵢ⁰ is the adsorption maximum amount; P is the gas vapor pressure, α_{AS} is the activity of a site occupied with an adsorbed gas A, α_{S} is an activity of the vacant site, γ₁and γ_{ϕ} are an activity coefficient of the occupied site with adsorbed gas component 1 and an activity coefficient of the vacant site, respectively.

12. The method of claim 11, further comprising reformulating Eq. 6, one obtains the following implicit adsorption isotherm expression:
wherein γ₁ and γ_{ϕ} are functions of x₁ and a relationship between the thermodynamic adsorption equilibrium constant K° and the apparent adsorption equilibrium constant K is shown in Eq. 8.
$$K ( {x}_{1} ) = K \text{?} \frac{\text{?}}{\text{?}} . \qquad ( 8 )$$
$$\text{?} \text{indicates text missing or illegible when filed}$$

13. The method of claim 11, further comprising calculating one or more pure component isotherms for gases with adsorbents including silica gels, activated carbons, zeolites and metal organic frameworks.

14. The method of claim 11, wherein the first temperature is a fixed temperature; or the first pressure is a relative pressure with a range of 0 to 0.1.

15. (canceled)

16. The method of claim 11, further comprising calculating one or more pure component isotherms for gases with adsorbents including silica gels, activated carbons, zeolites and metal organic frameworks at one or more temperatures.

17. The method of claim 11, wherein the site activities are further calculated with an adsorption Non-Random Two-Liquid (aNRTL) activity coefficient.

18. The method of claim 11, wherein a reference state for an occupied site with adsorbed gas component 1 is at full surface coverage and a saturated adsorption state is x₁=1.

19. The method of claim 11, further comprising substituting the species concentrations with the species activities and calculates the species activity coefficients with the adsorption Non-Random Two-Liquid activity coefficient.

20. The method of claim 11, wherein an adsorption equilibria calculated is at least one of: thermodynamically consistent; requires few adjustable model parameters; is applicable to both pure component adsorption isotherms and multicomponent adsorption isotherms; or calculates multicomponent adsorption isotherms from pure component adsorption isotherms .

21. A computerized method for thermodynamic formulation of a Langmuir isotherm comprising:
performing a calculation comprising:
$$n \text{?} = n \text{?} \frac{\text{?}}{\text{?}} \qquad ( 1 )$$
$$\text{?} \text{indicates text missing or illegible when filed}$$
wherein nᵢ is the adsorption amount of gas component i; nᵢ⁰ is the adsorption maximum amount; P is the gas vapor pressure, and K is the apparent adsorption equilibrium constant in which adsorption and desorption rates are proportional to a concentration of vacant sites and occupied sites; and
substituting the concentration of both a vacant site and an occupied site with site activities, wherein a reference state for the vacant sites is at zero surface coverage while the reference state for the occupied sites is at full surface coverage;
wherein the foregoing steps are performed by one or more processors.

22. The method of claim 21, further comprising substituting the constant K with a thermodynamic adsorption equilibrium constant K° calculated:
$$K \text{?} \text{?} \frac{\text{?}}{\text{?}} \text{?} \frac{\text{?}}{\text{?}} \text{?} \frac{\text{?}}{\text{?}}$$
$$\text{?} \text{indicates text missing or illegible when filed}$$
wherein α_{AS} is the activity of a site occupied with an adsorbed gas A, α_{S} is an activity of the vacant site, γ₁ and γ_{ϕ} are an activity coefficient of the occupied site with adsorbed gas component 1 and an activity coefficient of the vacant site, respectively.

23. The method of claim 21, wherein a system for classifying data comprises:
at least one input/output interface;
a data storage;
one or more processors communicably coupled to the at least one input/output interface and the data storage, wherein the one or more processors perform the step of:
determining adsorption isotherms for at least one of a first temperature, a first pressure, a low temperature, or a high pressure region, or both comprising:
$$K \text{?} \text{?} \frac{k \text{?}}{k \text{?}} \text{?} \frac{\text{?}}{\text{?}} \text{?} \frac{\text{?}}{\text{?}} \qquad ( 6 )$$
$$\text{?} \text{indicates text missing or illegible when filed}$$
wherein α_{AS} is the activity of a site occupied with an adsorbed gas A, α_{S} is an activity of the vacant site, γ₁ and γ_{ϕ} are an activity coefficient of the occupied site with adsorbed gas component 1 and an activity coefficient of the vacant site, respectively; and
receiving the data from the at least one input/output interface.

24. A computer program embodied on a non-transitory computer readable storage medium that is executed using one or more processors for thermodynamic formulation of a Langmuir isotherm comprising:
(a) a code segment for receiving data to calculate the Langmuir isotherm;
(b) a code segment for determining adsorption isotherms for at least one of a first temperature, a first pressure, a low temperature, or a high pressure region, or both comprising:
$$K \text{?} \text{?} \frac{k \text{?}}{k \text{?}} \text{?} \frac{\text{?}}{\text{?}} \text{?} \frac{\text{?}}{\text{?}} \qquad ( 6 )$$
$$\text{?} \text{indicates text missing or illegible when filed}$$
wherein a_{AS} is the activity of a site occupied with an adsorbed gas A, a_{S} is an activity of the vacant site, γ₁ and γ_{ϕ} are an activity coefficient of the occupied site with adsorbed gas component 1 and an activity coefficient of the vacant site, respectively; and
(c) a code segment for outputting the data from at least one input/output interface.
